(19) United States
(12) Patent Application Publication
(10) Pub. No.: US 2005/0112069 A1
(43) Pub. Date: May 26, 2005

(54) Pharmaceutical composition of a pde4 or pde 3/4 inhibitor and histamine receptor antagonist

(76) Inventors: Rolf Beume, Konstanz (DE); Daniela Bundschuh, Konstanz (DE); Christian Weimar, Konstanz (DE); Stefan-Lutz Wollin, Meersburg (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/506,875

(22) PCT Filed: Feb. 25, 2003
(86) PCT No.: PCT/EP03/01876

(30) Foreign Application Priority Data
Mar. 6, 2002 (EP) 02004987.0

(51) Int. Cl. A61L 9/04; A61K 9/14; A61K 31/551; A61K 31/522; A61K 31/4439

(52) U.S. Cl. 424/46; 514/220; 514/263.34; 514/406; 514/337; 514/291; 514/314

(57) ABSTRACT

The invention relates to the combined administration of PDE4 or PDE3/4 inhibitors and histamine receptor antagonists for the treatment of respiratory diseases.

### FIELD OF APPLICATION OF THE INVENTION

The present invention relates to combinations of pharmaceutically active substances for use in the treatment of respiratory diseases.

The substances used in the combinations according to the invention are known active compounds from the PDE4 and PDE3/4 inhibitors class and active compounds from the class of the histamine receptor antagonists.

### KNOWN TECHNICAL BACKGROUND

In the international application WO03/000289 compositions are described which comprise a PDE4 inhibitor and a H1-receptor antagonist and the use of these compositions for the manufacture of a medicament for the treatment of respiratory diseases.

### DESCRIPTION OF THE INVENTION

Asthma is a common inflammatory disease of the respiratory tract, accounting for 1-3% of all office visits, 500,000 hospital admissions per year and more pediatric hospital admissions than any other single illness in the US. Annually, more than 5000 children and adults die of asthma attacks in the United States (William E. S.; Goodmann Gilmann A.:The pharmacological Basis of Therapeutics, 9^{th} Edition, pp. 152 & 659-682, Mc Graw Hill, New York 1996).

Asthma can no longer be viewed simply as a reversible airway obstruction. It should instead be considered primarily as an inflammatory illness that has bronchial hyperactivity and bronchospasm as its results. Allergen specific immunoglobulin E (IgE) is bound to the mast cells via Fc receptors. It is a fragment obtained by papain digestion of immunoglobulin molecules and contains most of the antigenic determinants. When an allergen comes into contact with IgE, the mast cells are activated and release a number of inflammatory mediators, which include granule contents like histamine, proteases, heparin, and tumor necrosis factor (TNF), a variety of lipid membrane derived molecules like prostaglandins, leukotrienes and platelet activating factor (PAF), and a number of cytokines like interleukin (IL)-1, 3, 4, 5, 6 and 8 and chemokines. An enormous variety of mediators are released which have more than one potent effect on airway inflammation.

As a result of vasodilation, increased vasopermeability and increased endothelial adhesiveness towards leukocytes further leads to an influx of inflammatory cells like lymphocytes, eosinophils and macrophages from blood circulation into the tissues. This in turn leads to the release of mediators which have further inflammatory effects (Rao A. R. et al. Recent Perspectives in the design of antiasthmatic agents, Pharmazie, 55, 7, 475-482, 2000).

Thus, it can be understood that it is unlikely that drugs affecting a single mediator can satisfactorily treat the disease alone. As asthma is one of the major diseases affecting mankind, there is a need to develop drugs which can affect a wide variety of mediators.

Therefore, it is the object of the present invention to make available respiratory tract therapeutics which fulfill the following conditions:
Favorable simultaneous influence on several of the inflammatory mediators
Marked bronchorelaxation and -dilatation
Good oral availability
Minor side effects
Good suitability for long-term therapy
Favorable influence on bronchial hyperreactivity

It has now been found that the combined use of a PDE4 or a PDE3/4 inhibitor and a histamine receptor antagonist outstandingly fulfills the above-mentioned conditions.

The invention thus relates to the combined use of a PDE4 or a PDE3/4 inhibitor and a histamine receptor antagonist in the treatment of respiratory diseases.

By the expression “PDE4 inhibitor” is meant a selective phosphodiesterase inhibitor, which inhibits preferentially the type 4 phosphodiesterase when compared to other known types of phosphodiesterase, e.g. type 1, 2, 3, 5 etc., whereby the compound has a lower IC₅₀ (more potent) for the type 4 phosphodiesterase, such as where the IC₅₀ for PDE4 inhibition is about factor 10 lower compared to IC₅₀ for Inhibition of other known type of phosphodiesterase, e.g. type 1, 2, 3, 5 etc.

Analogously, the expression “PDE3/4 inhibitor” is defined. Methods to determine the activity and selectivity of a phosphodiesterase inhibitor are known to the person skilled in the art. In this connection it may be mentioned, for example, the methods described by Thompson et al. (Adv Cycl Nucl Res 10: 69-92, 1979), Glembycz et al. (Br J Pharmacol 118: 1945-1958,1996) and the phosphodiesterase scintillation proximity assay of Amersham Pharmacia Biotech.

By the expression “histamine receptor antagonist” are meant H₁-receptor antagonists, particularly the so called H₁-receptor antagonists of the second generation (Mutschler, Arzneimittelwirkungen, 8. Edition, 2001, pages 456-461).

As possible PDE4 or PDE3/4 inhibitors within the meaning of the present invention may be mentioned, by way of example, those PDE4 or PDE3/4 inhibitors which are named expressis verbis as an example, or described or claimed generically in the following patent applications and patents: DE 1545687, DE 2028869, DE 2123328, DE 2315801, DE 2402908, DE 2413935, DE 3900233, EP 0103497, EP 0139464, EP 0158380, EP 0163965, EP 0335386, EP 0389282, EP 0393500, EP 0428302, EP 0435811, EP 0449216, EP 0459505, EP 0470805, EP 0490823, EP 0506194, EP 0510562, EP 0511865, EP 0527117, EP 0553174, EP 0557016, EP 0626939, EP 0664289, EP 0671389, EP 0685474, EP 0685475, EP 0685479, EP 0731099, EP 0736532, EP 0738715, EP 0748805, EP 0763534, EP 0816357, EP 0819688, EP 0819689, EP 0832886, EP 0834508, EP 0848000, JP 92234389, JP 94329652, JP 95010875, JP 98072415, JP 98147585, U.S. Pat. Nos. 5,703,098, 5,739,144, WO 9117991, WO 9200968, WO 9212961, WO 9307146, WO 9315044, WO 9315045, WO 9318024, WO 9319068, WO 9319720, WO 9319747, WO 9319749, WO 9319751, WO 9325517, WO 9402465, WO 9412461, WO 9420455, WO 9422852, WO 9427947, WO 9500516, WO 9501338, WO 9501980, WO 9503794, WO 9504045, WO 9504046, WO 9505386, WO 9508534, WO 9509623, WO 9509624, WO 9509627, WO 9509836, WO 9514667, WO 9514680, WO 9514681, WO 9517392, WO 9517399, WO 9519362, WO 9520578, WO 9522520, WO 9524381, WO 9527692, WO 9535281, WO 9535283, WO 9535284, WO 9600218, WO 9601825, WO 9606843, WO 9603399, WO 9611690, WO 9611917, WO 9612720, WO 9631486, WO 9631487, WO 9635683, WO 9636595, WO 9636596, WO 9636611, WO 9636625, WO 9636626, WO 9636638, WO 9638150, WO 9639408, WO 9640636, WO 9703967, WO 9704779, WO 9705105, WO 9708143, WO 9709345, WO 9712895, WO 9718208, WO 9719078, WO 9720833, WO 9722585, WO 9722586, WO 9723457, WO 9723460, WO 9723461, WO 9724117, WO 9724355, WO 9725312, WO 9728131, WO 9730999, WO 9731000, WO 9732853, WO 9735854, WO 9736905, WO 9740032, WO 9743288, WO 9744036, WO 9744322, WO 9747604, WO 9748697, WO 9804534, WO 9805327, WO 9806692, WO 9806704, WO 9807715, WO 9808828, WO 9808830, WO 9808841, WO 9808844, WO 9809946, WO 9809961, WO 9811113, WO 9814448, WO 9818796, WO 9821207, WO 9821208, WO 9821209, WO 9822453, WO 9831674, WO 9840382, WO 9845268, WO 9855481, WO 9856756, WO 9905111, WO 9905112, WO 9505113, WO 9906404, WO 9918095, WO 9931071, WO 9931090, WO 9947505, WO 9957115, WO 9957118, WO 9964414, WO 0001695, WO 0012501, WO 0026208, WO 0042017, WO 0042018, WO 0042019, WO 0042020, WO 0042034, WO 0119818, WO 0130766, WO 0130777, WO 0151470, WO 02006270, WO 02066476, WO 02064584 and WO 02085885.

Exemplary PDE inhibitors are shown on the following pages with the aid of their formulae:

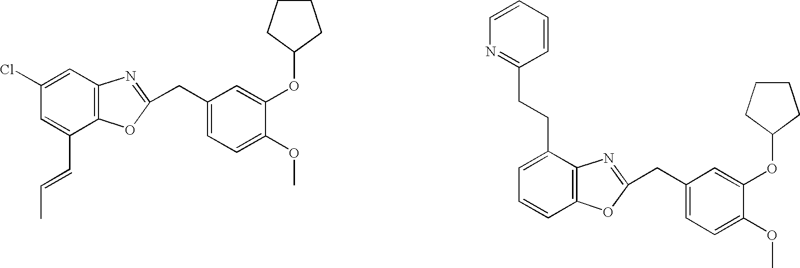

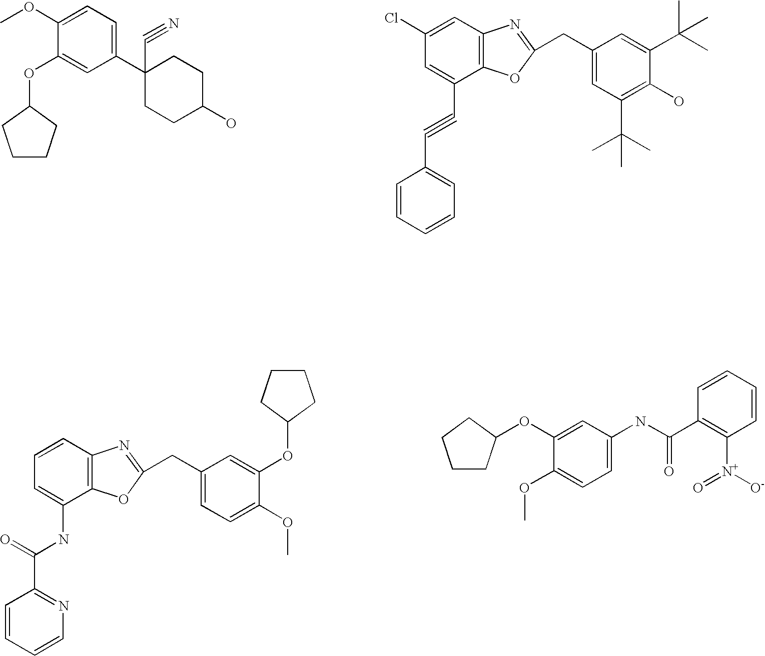

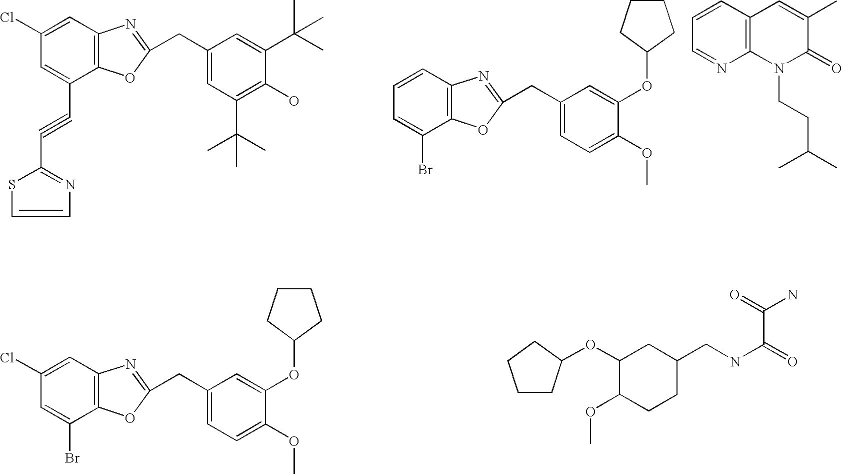

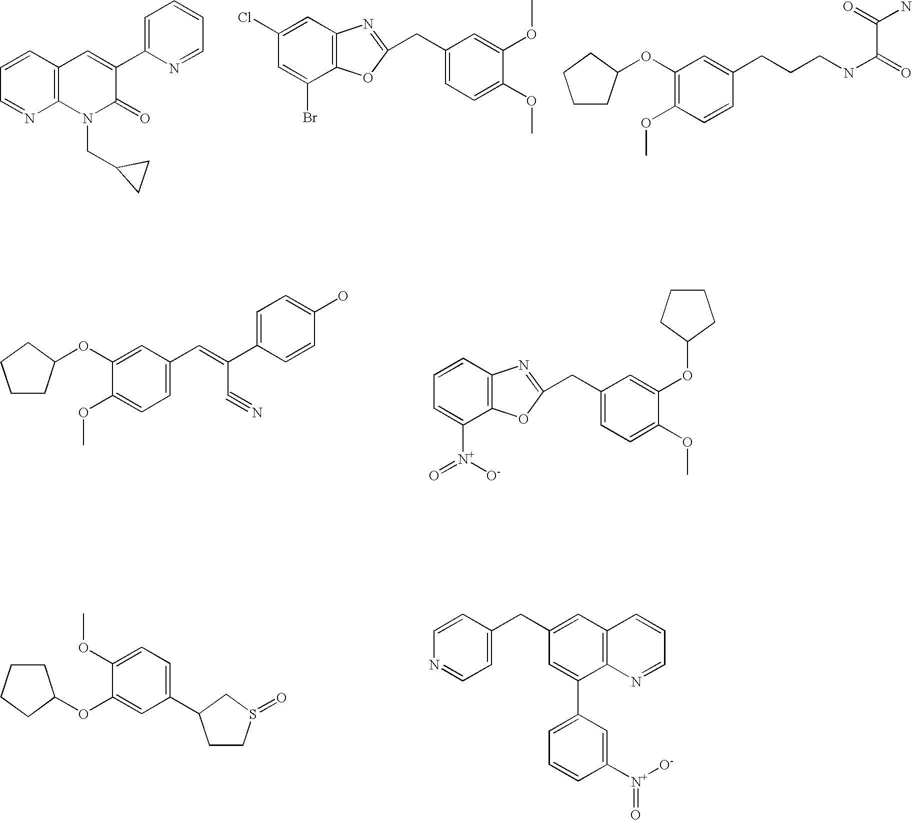

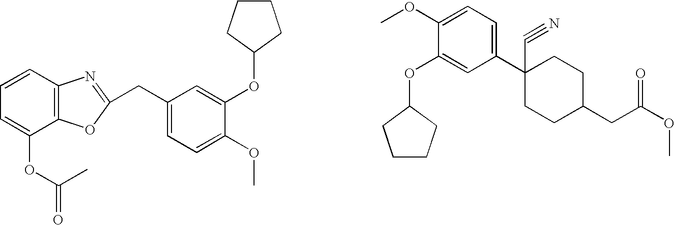

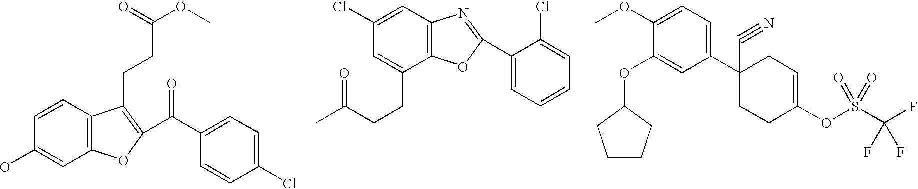

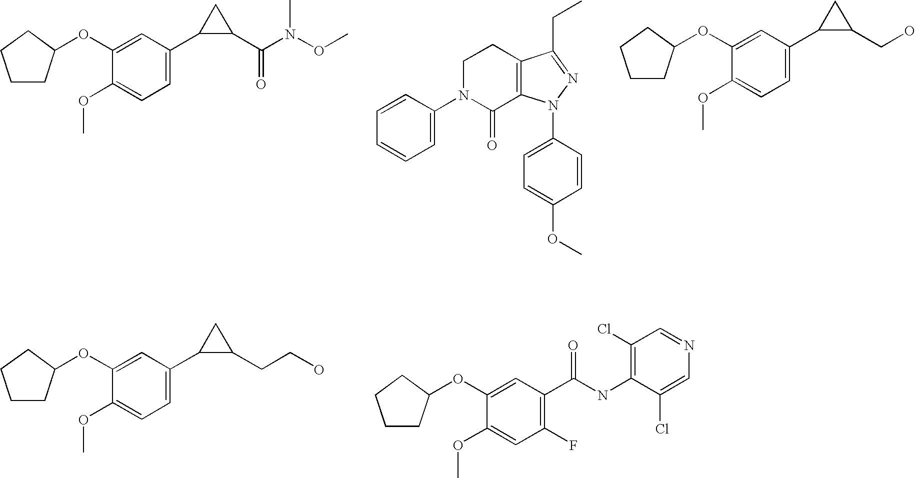

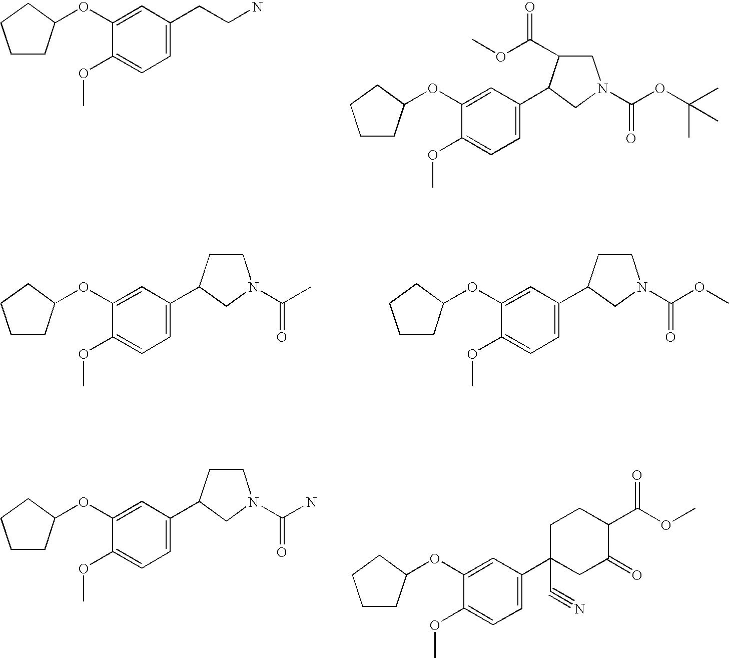

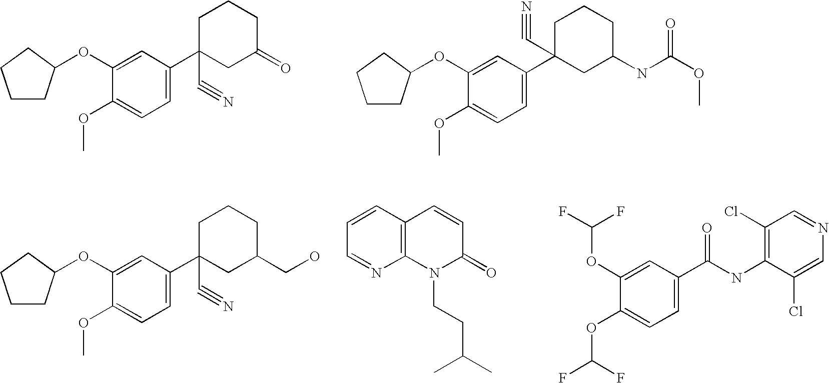

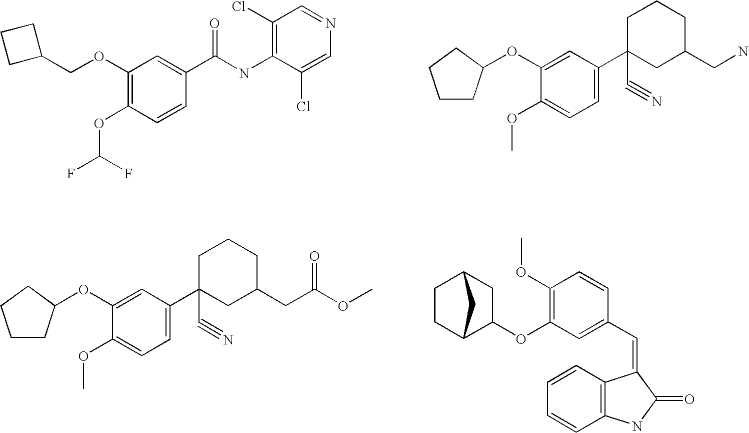

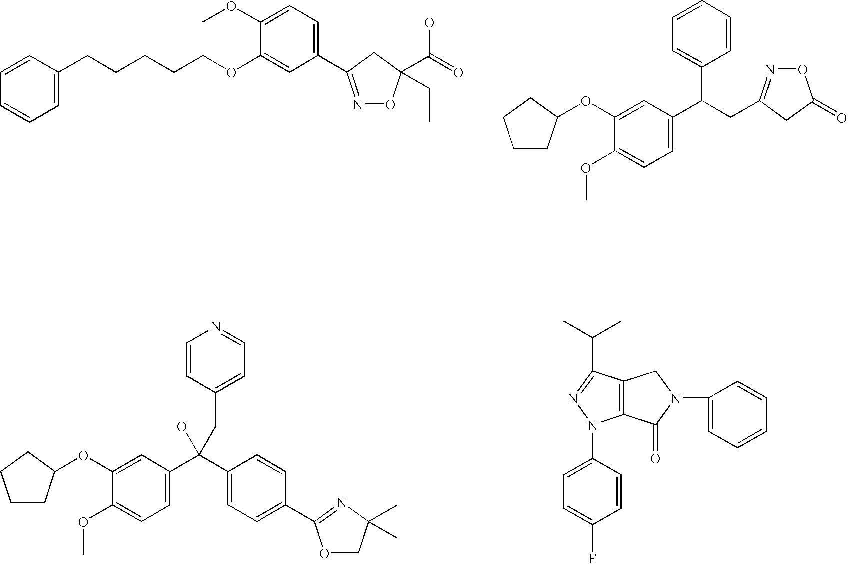

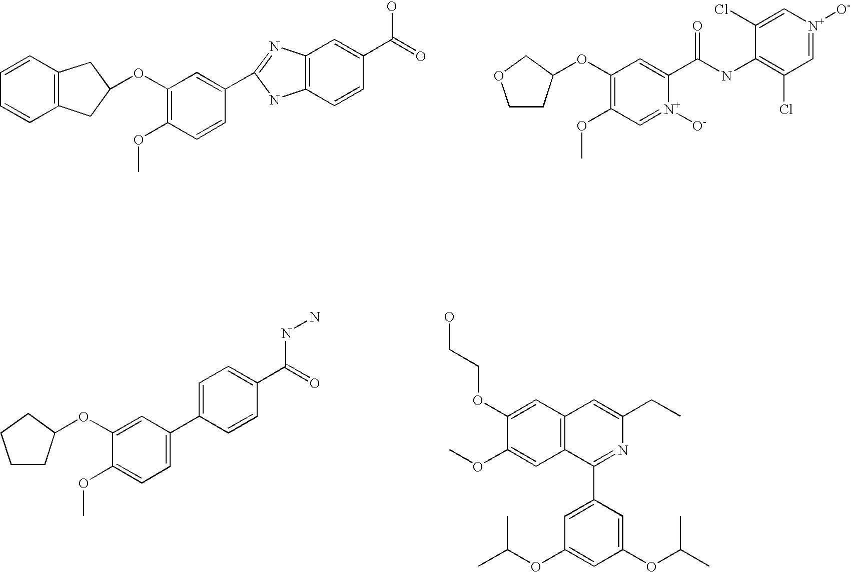

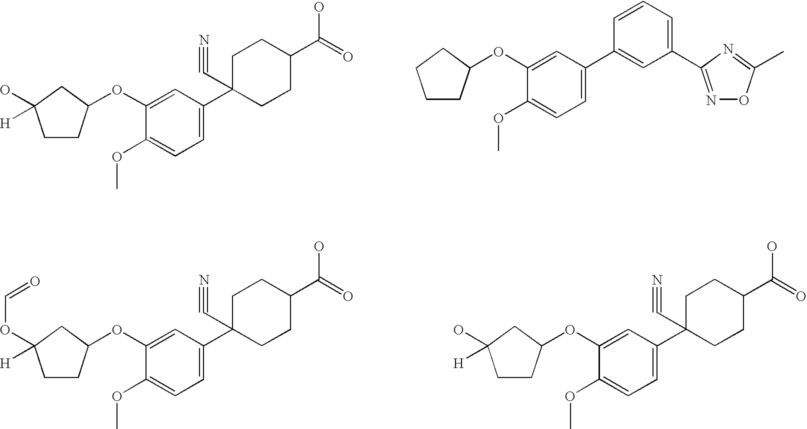

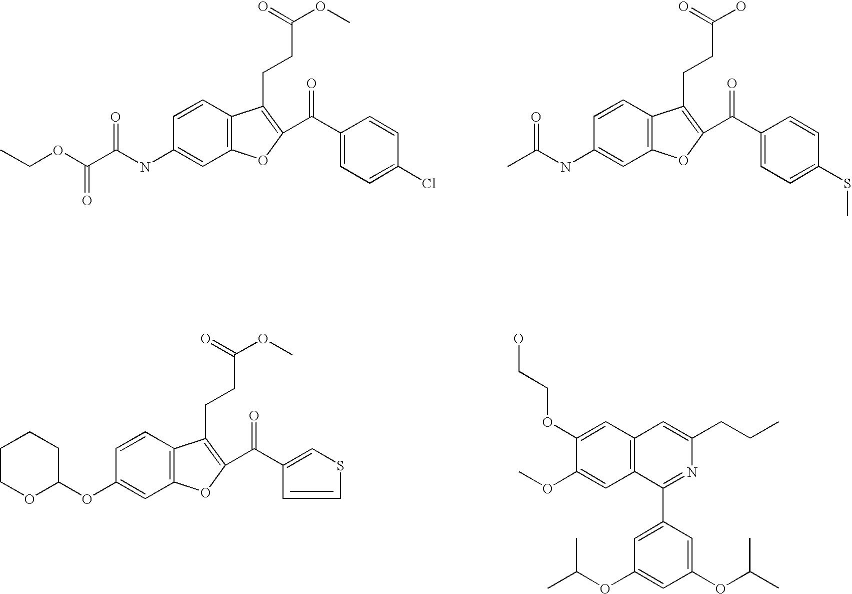

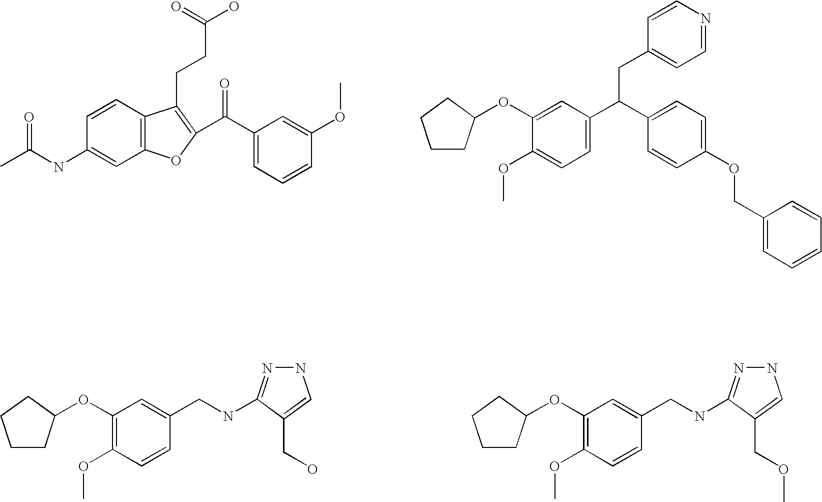

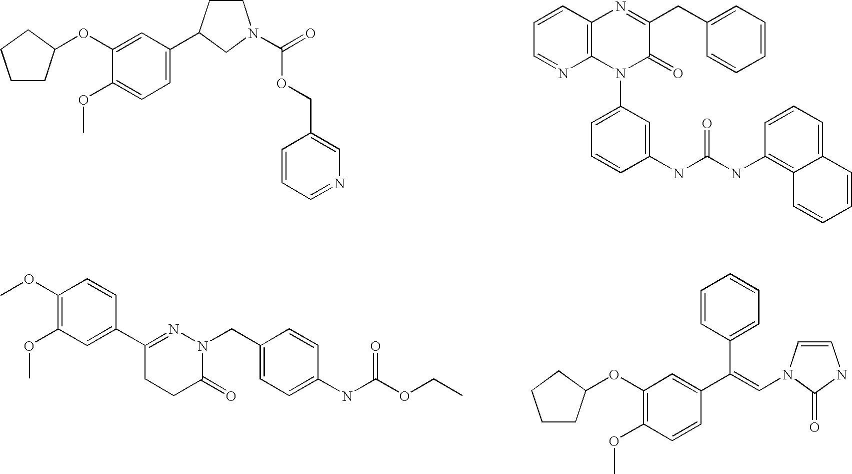

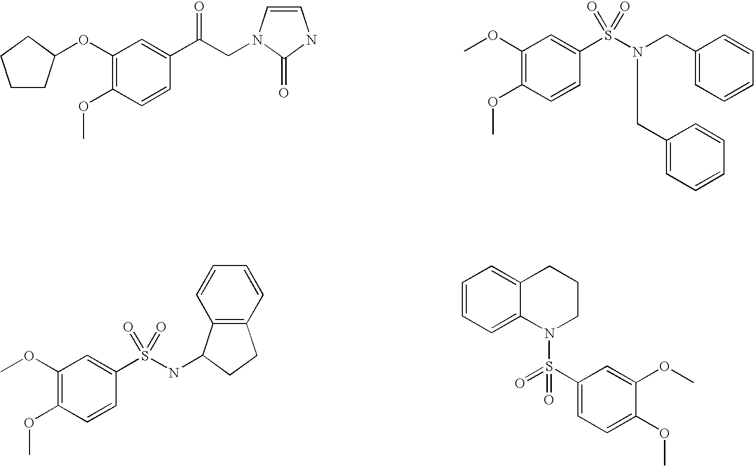

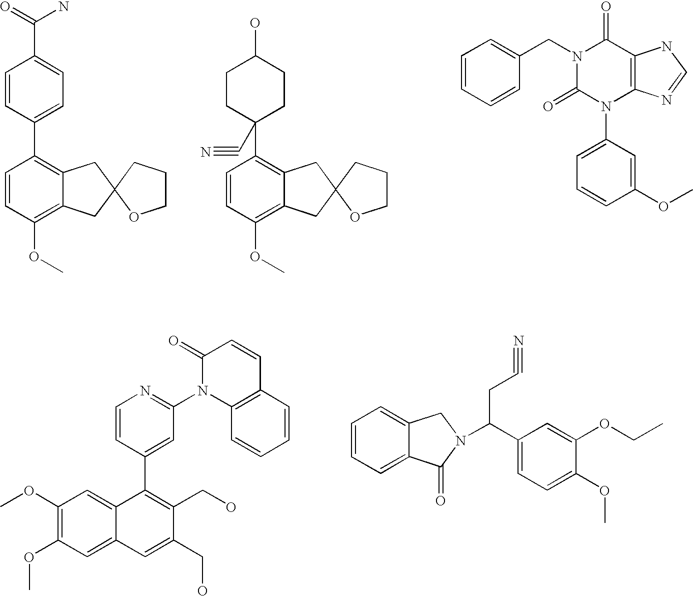

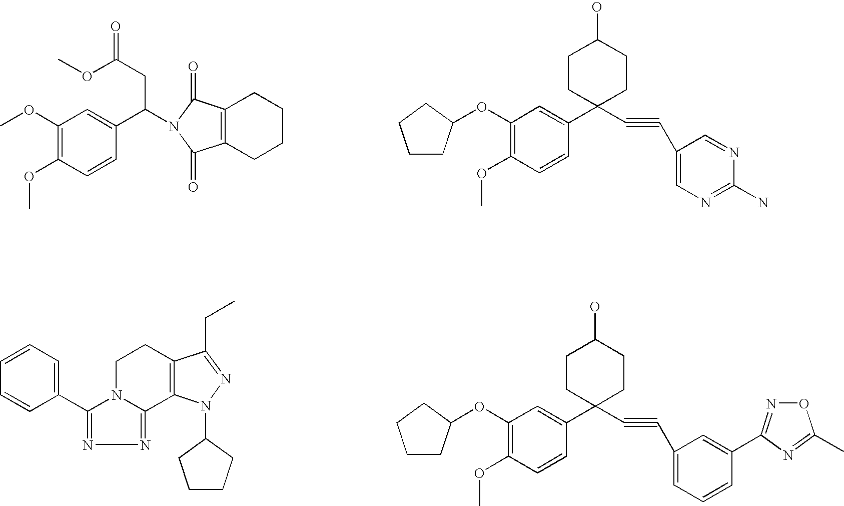

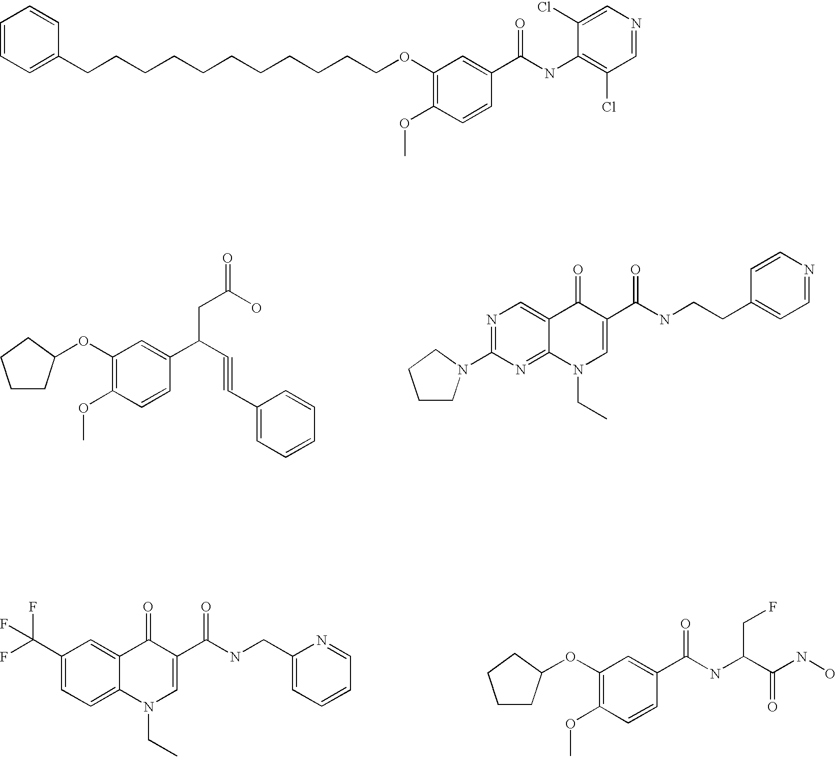

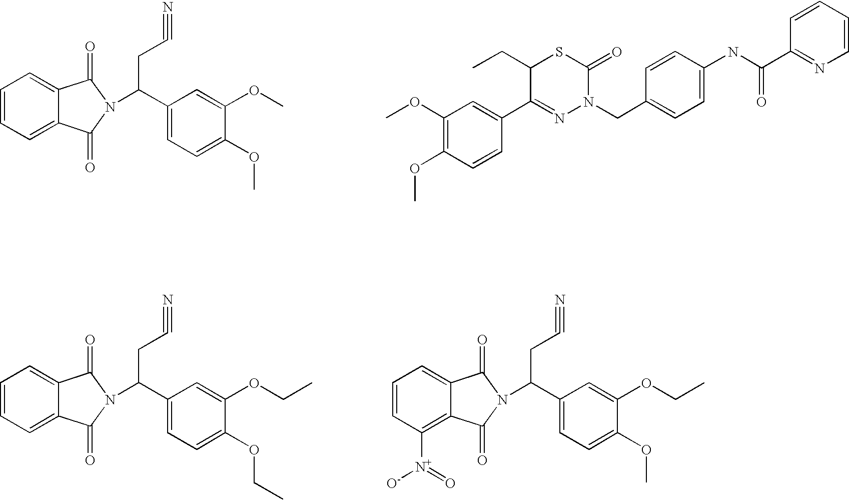

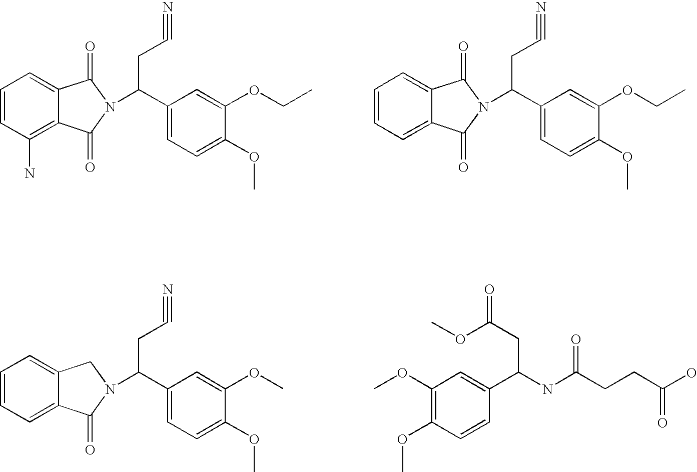

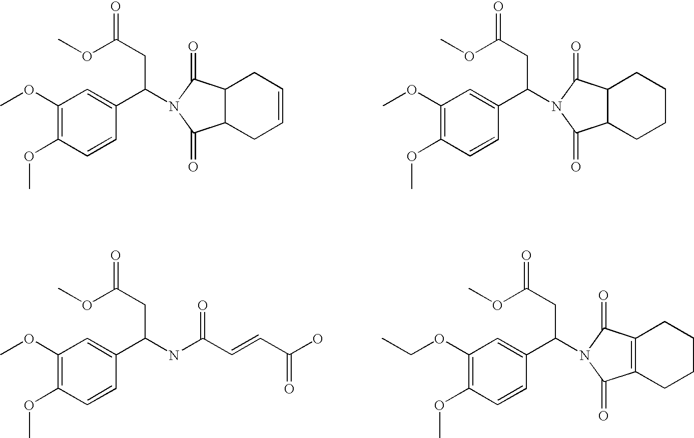

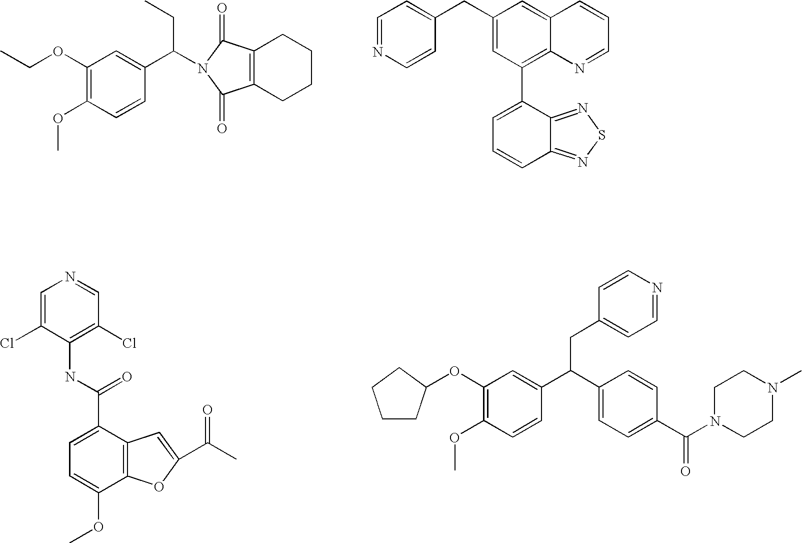

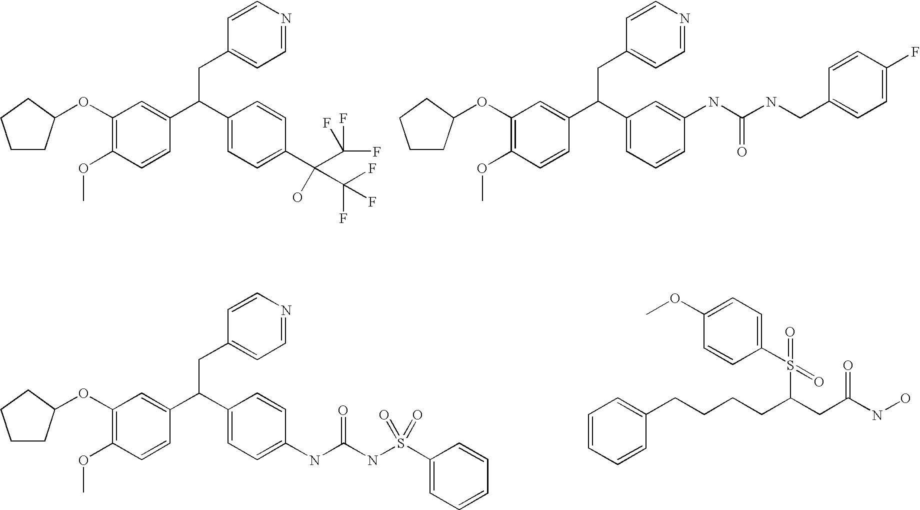

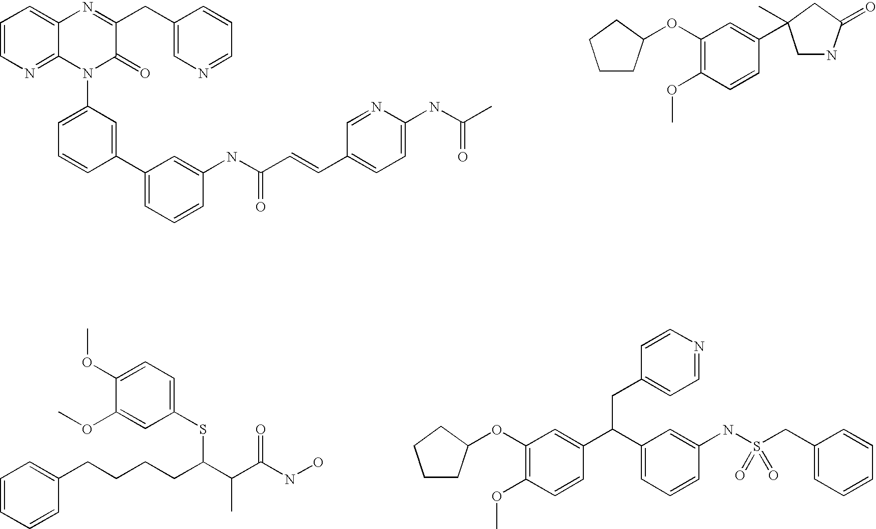

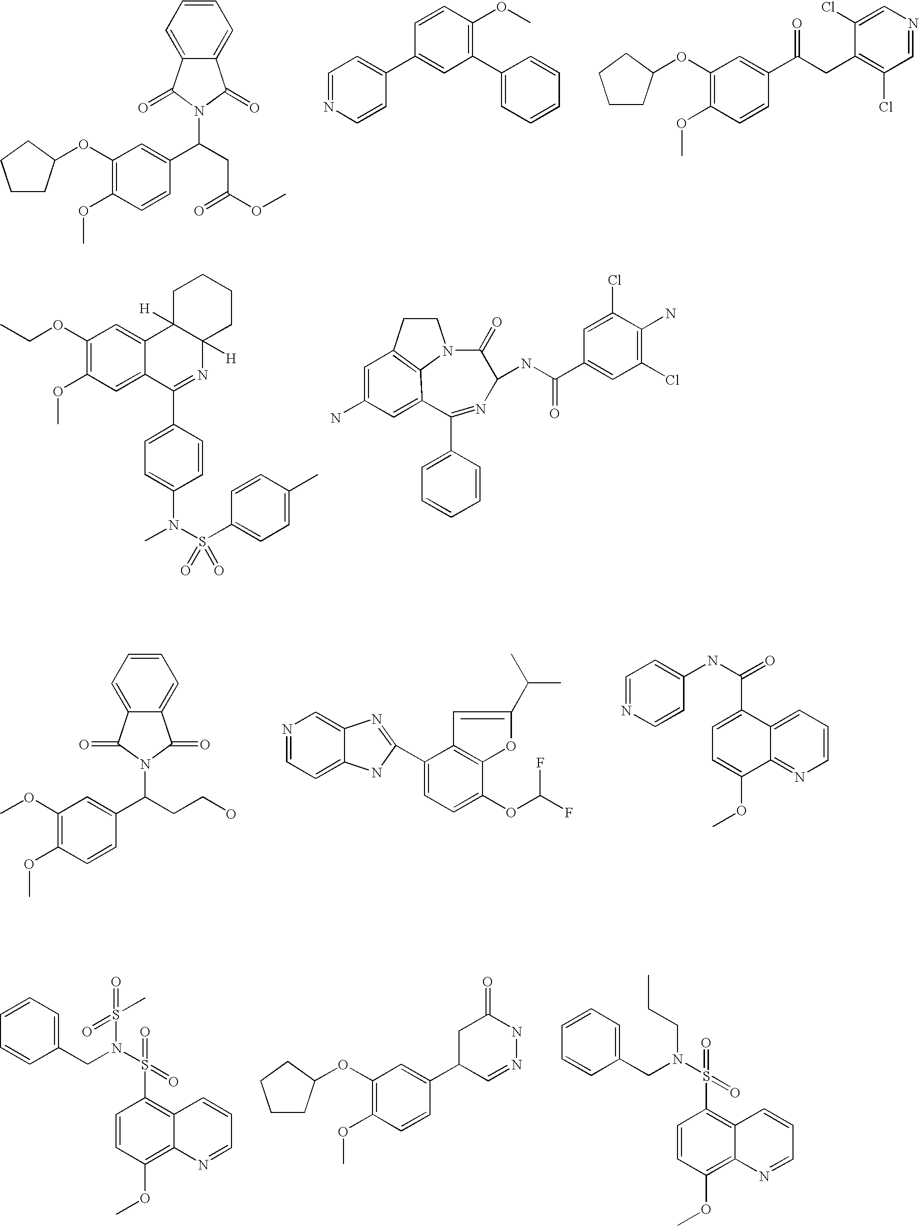

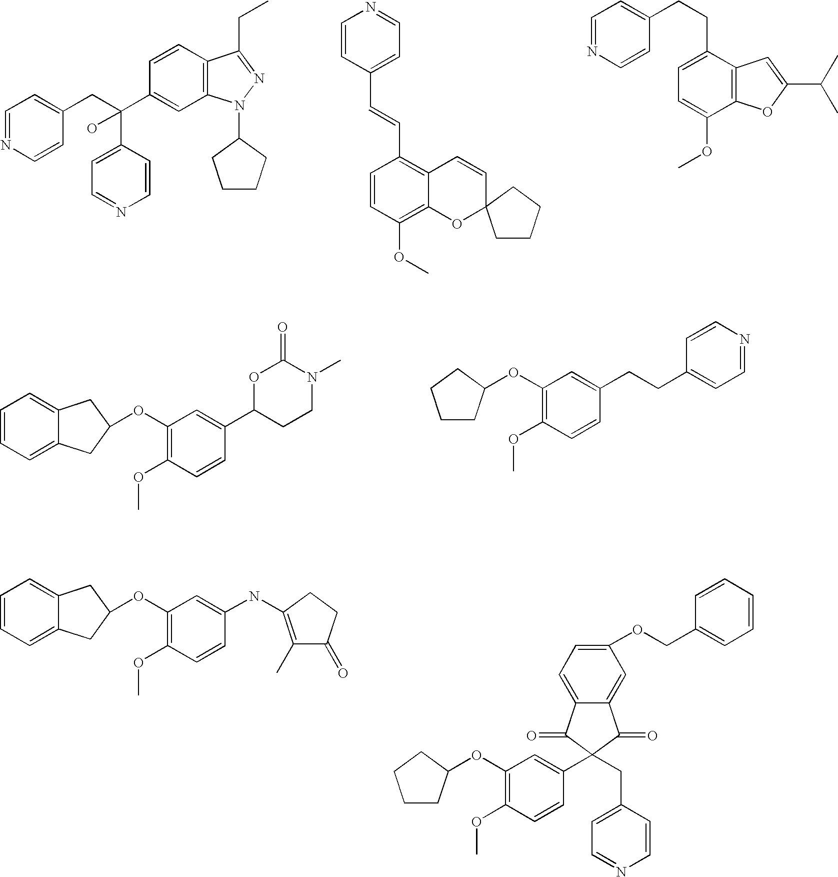

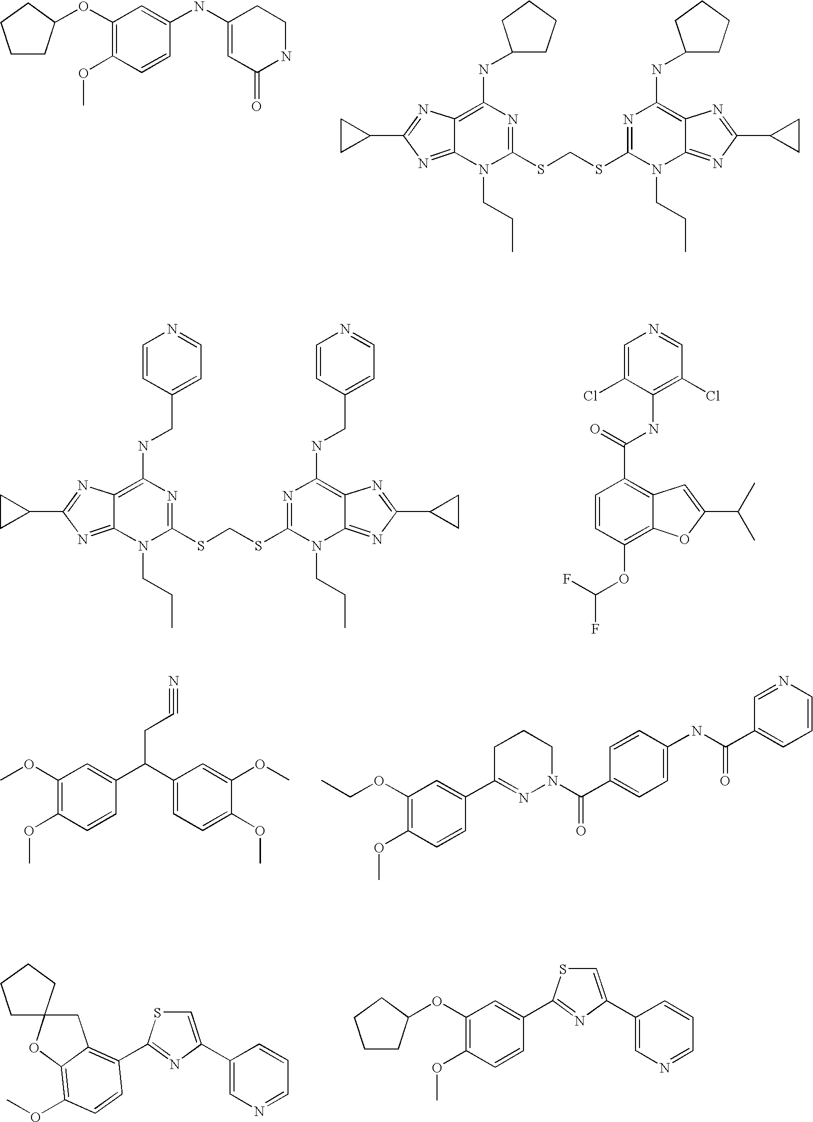

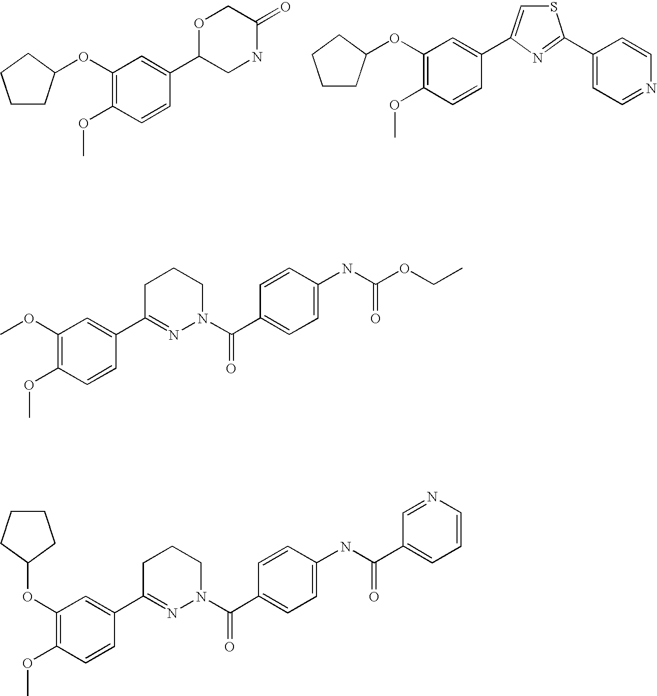

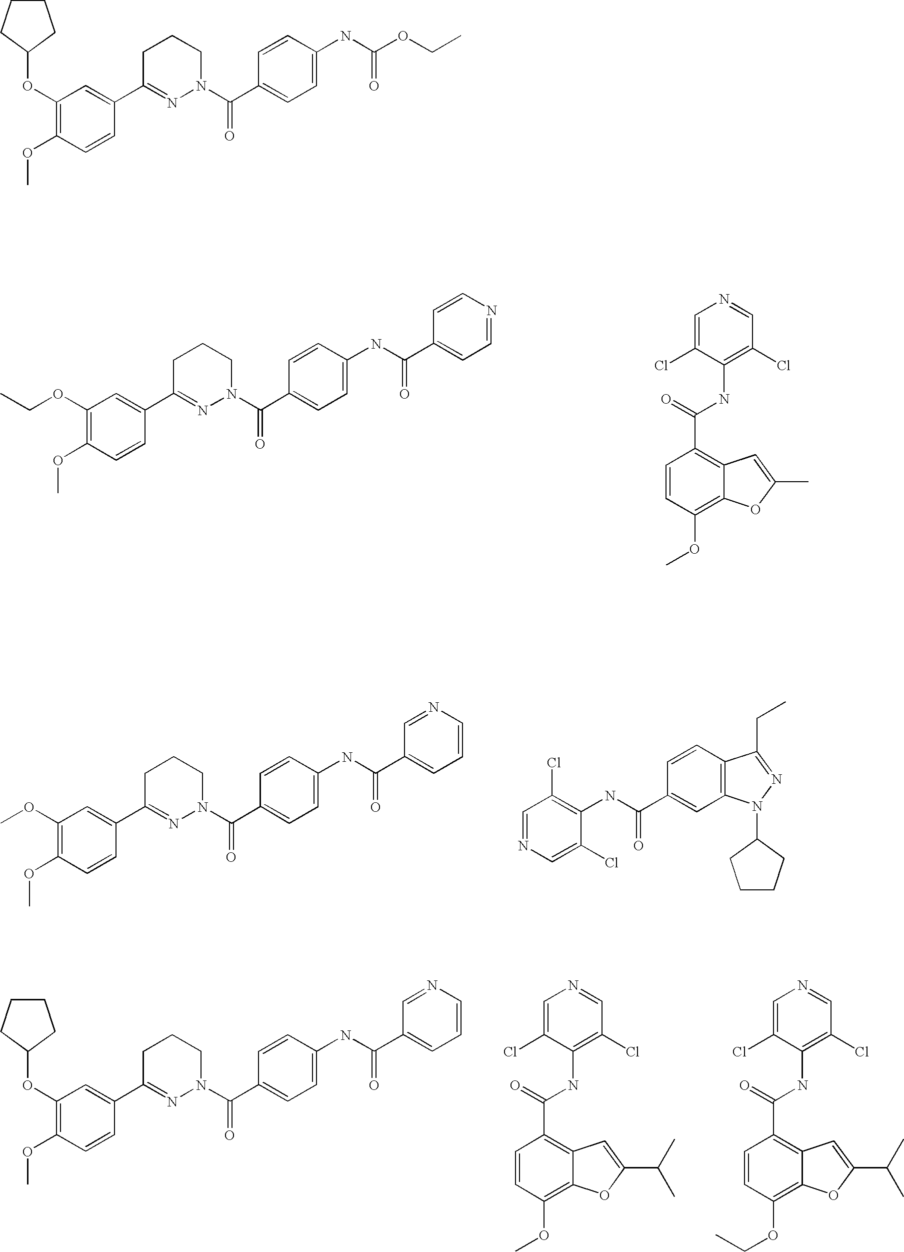

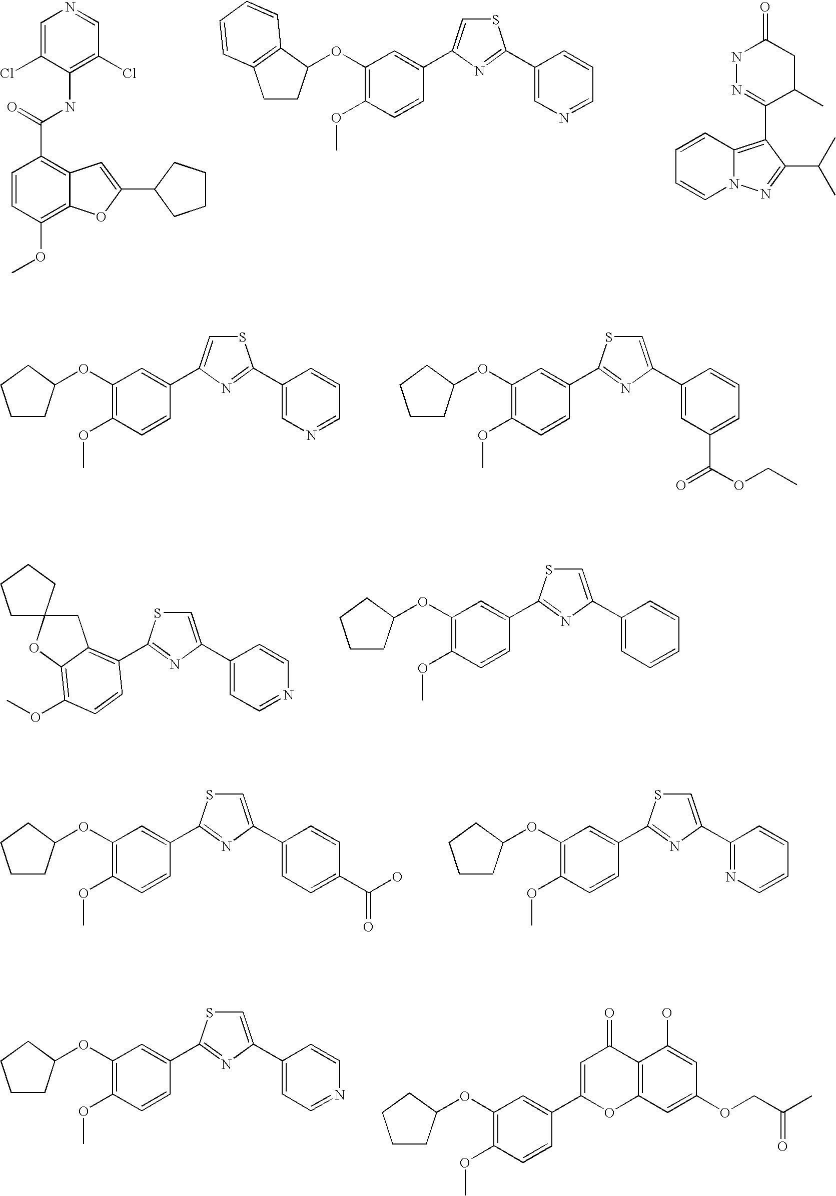

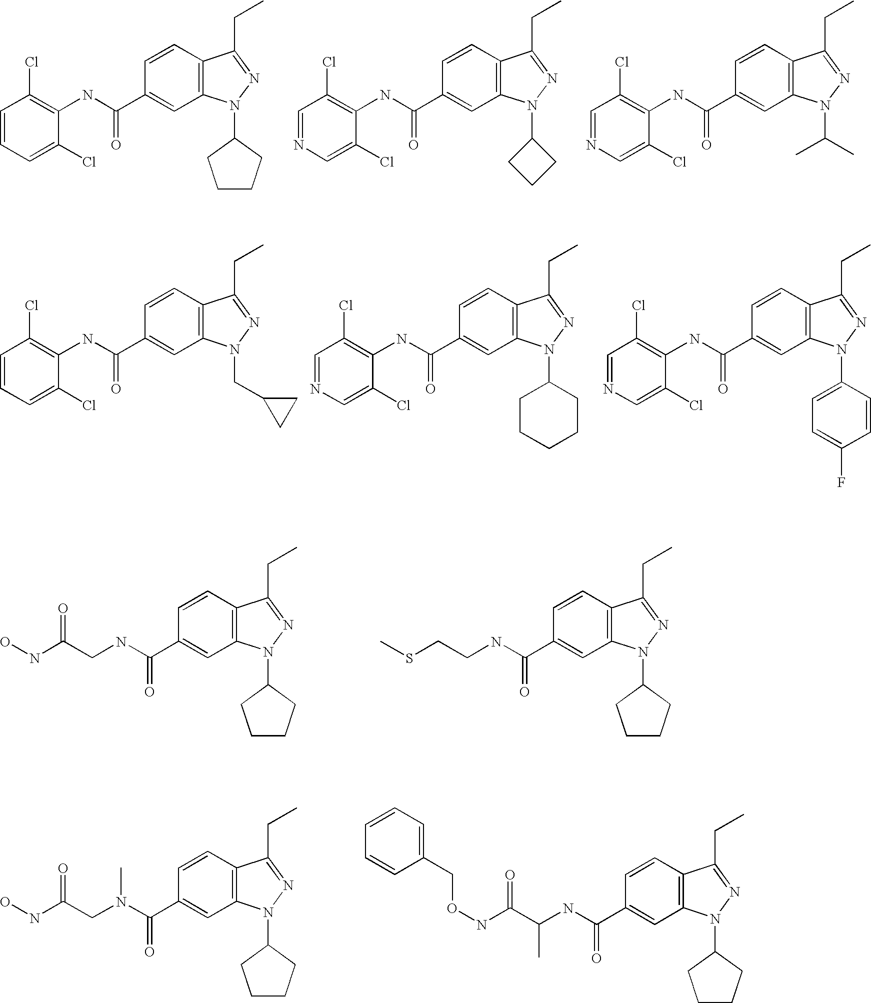

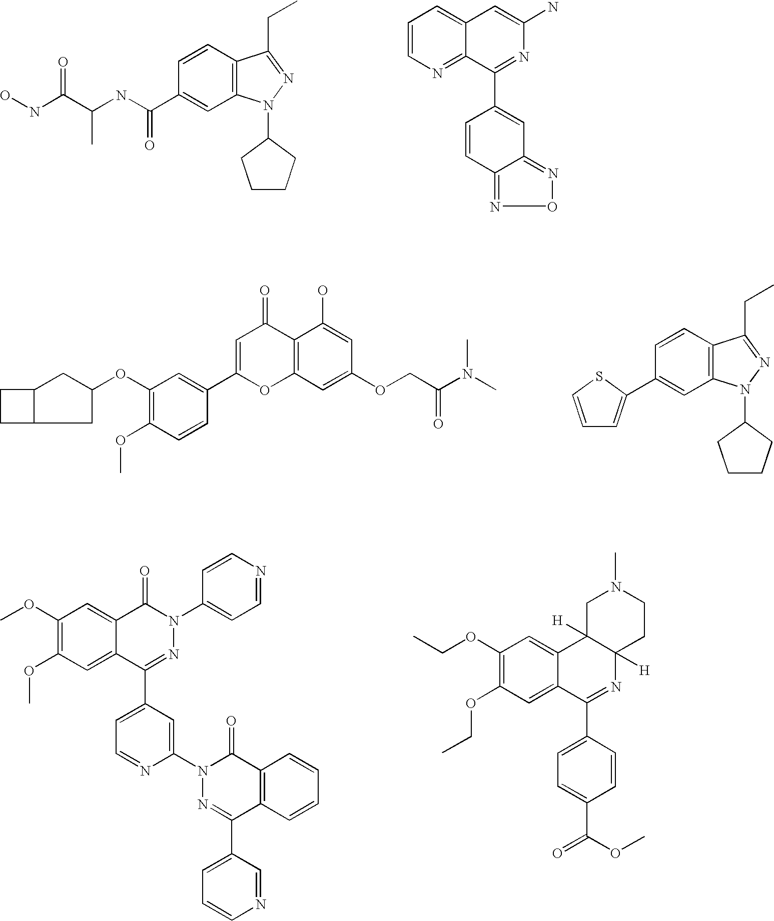

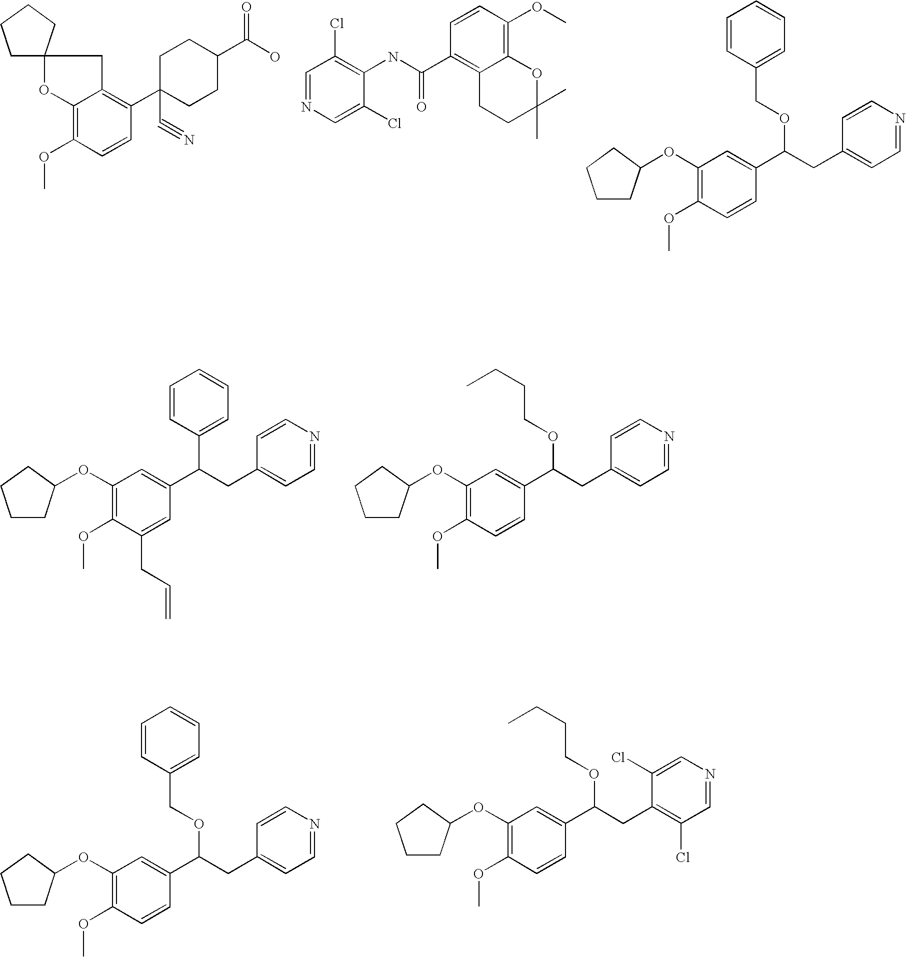

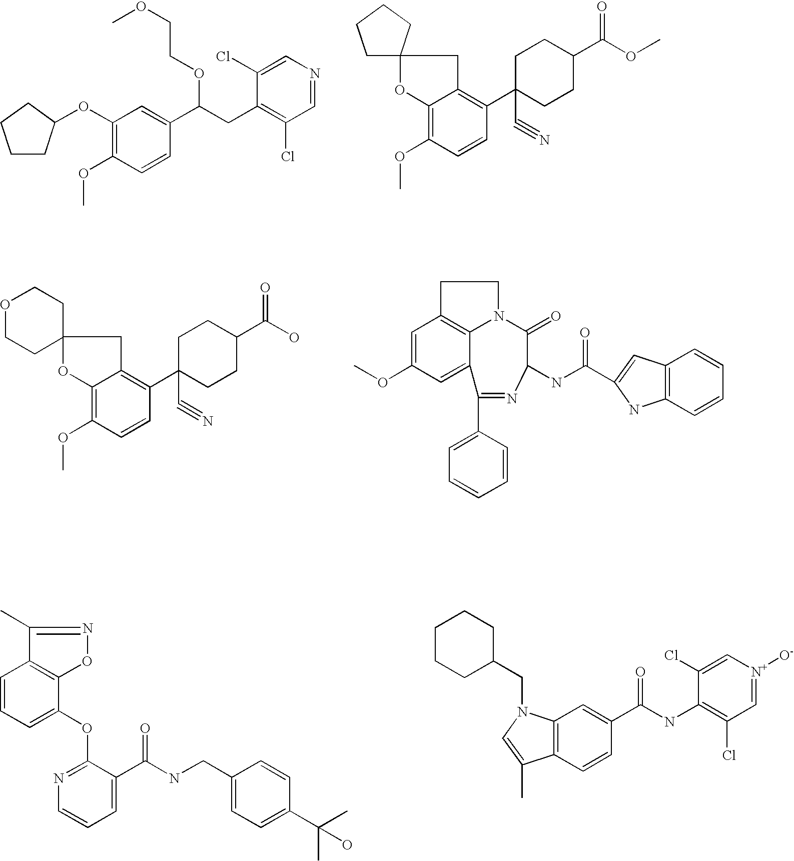

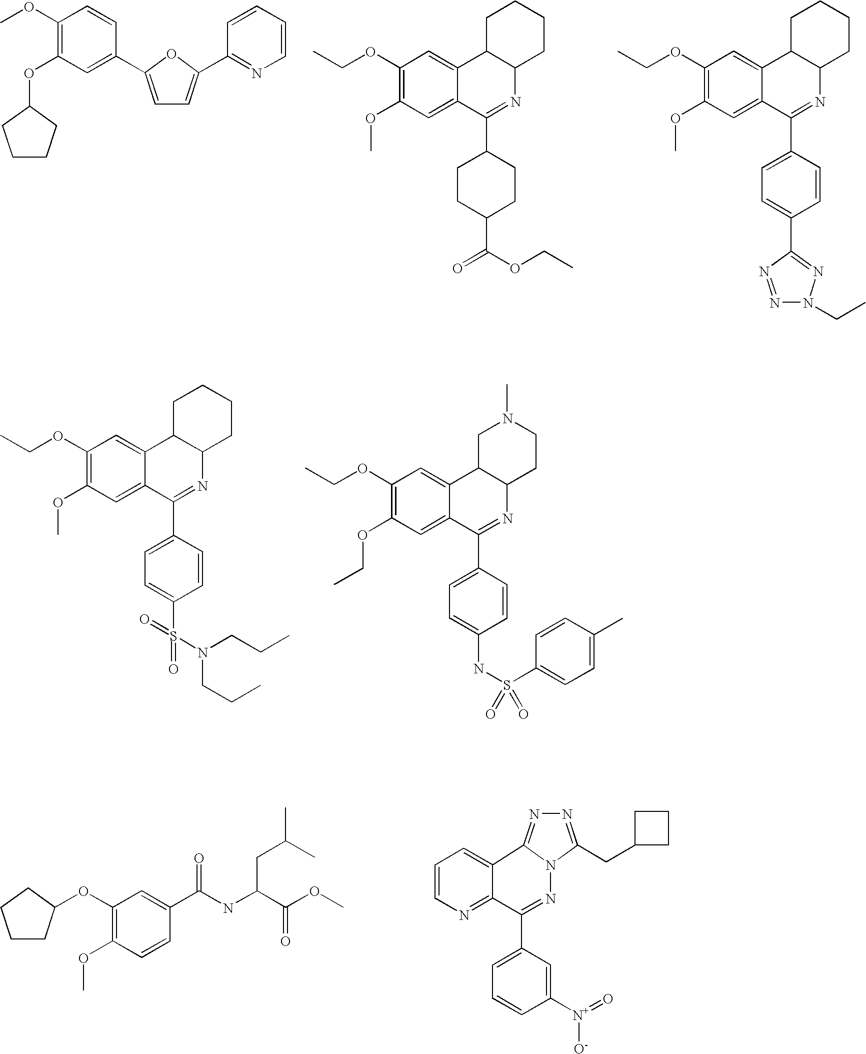

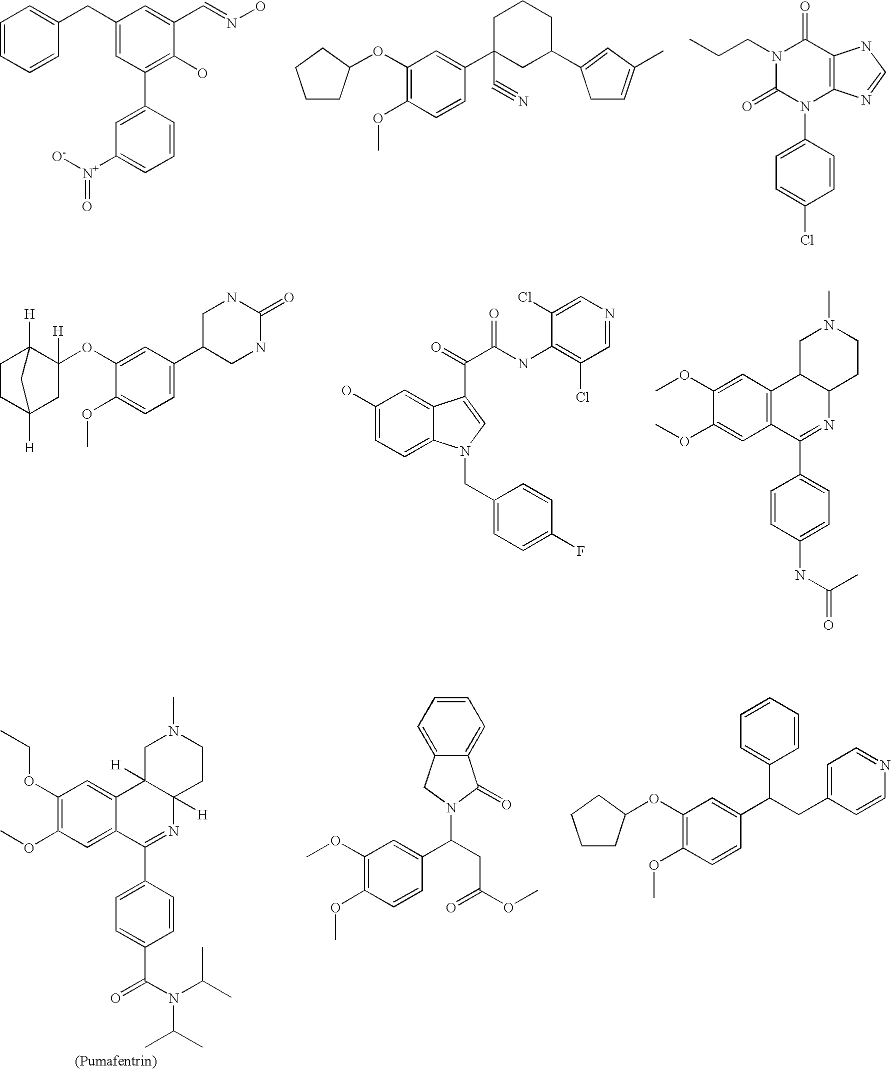

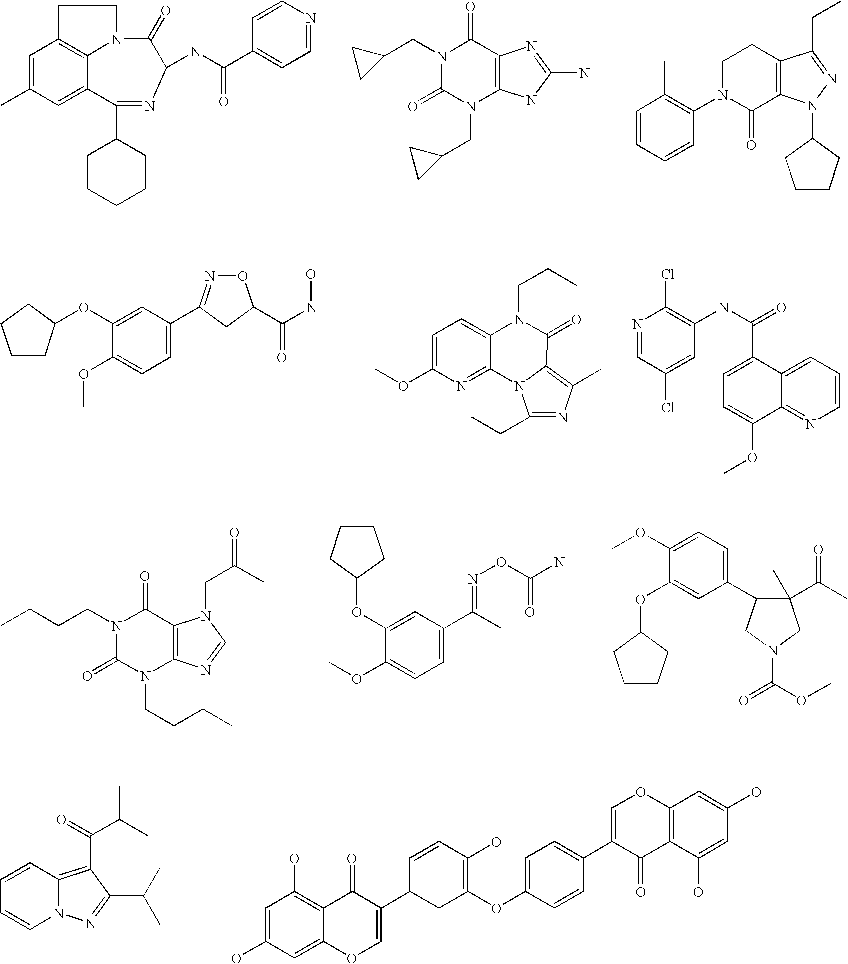

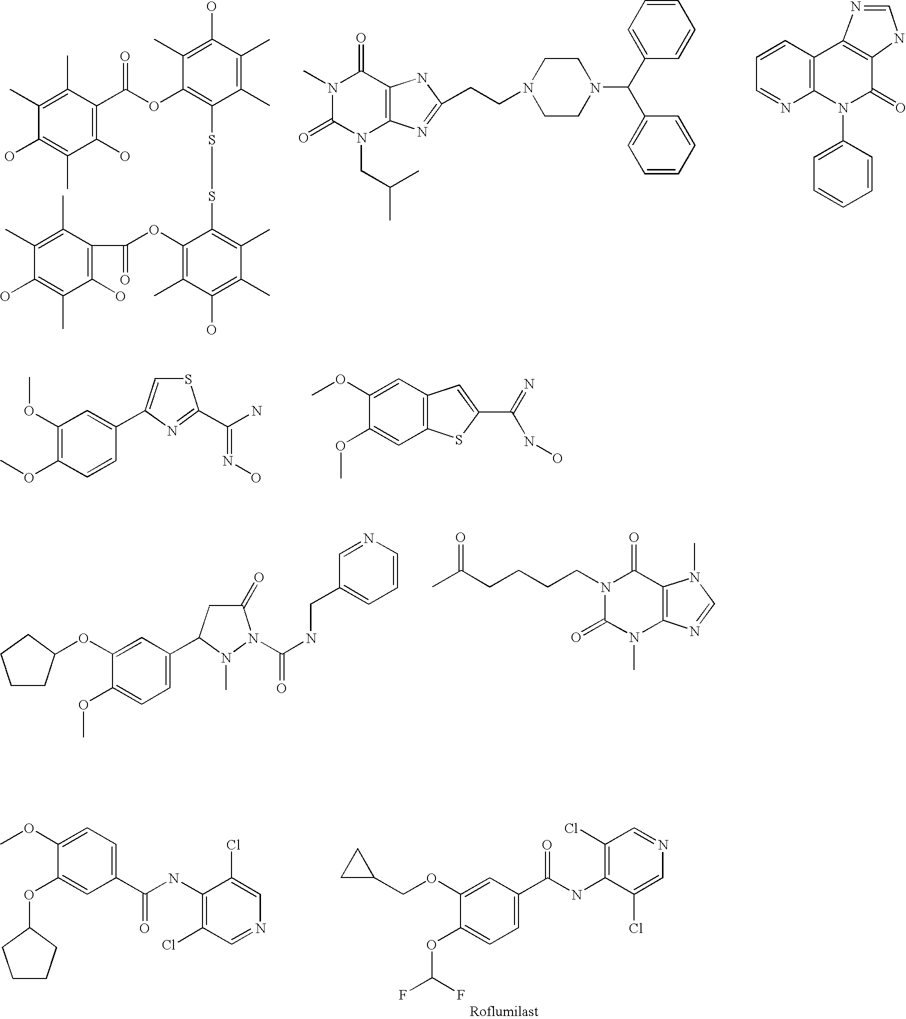

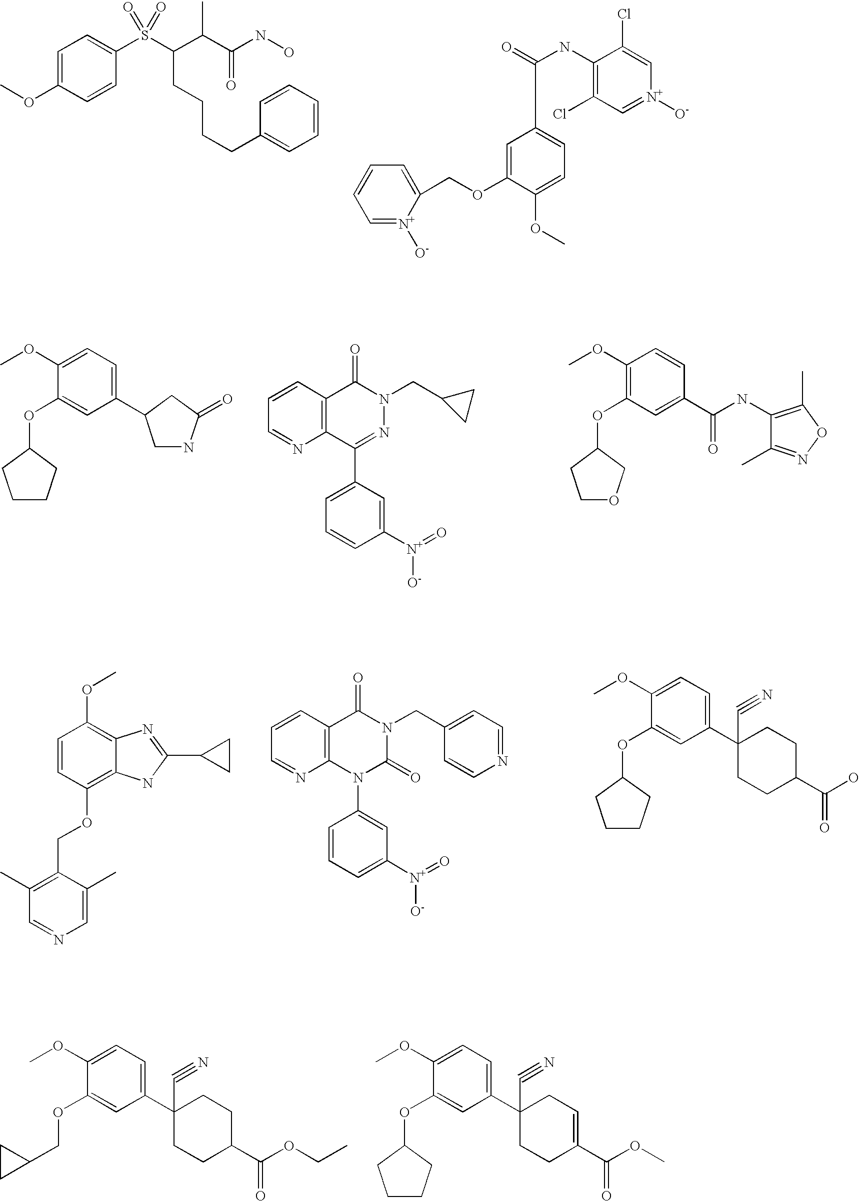

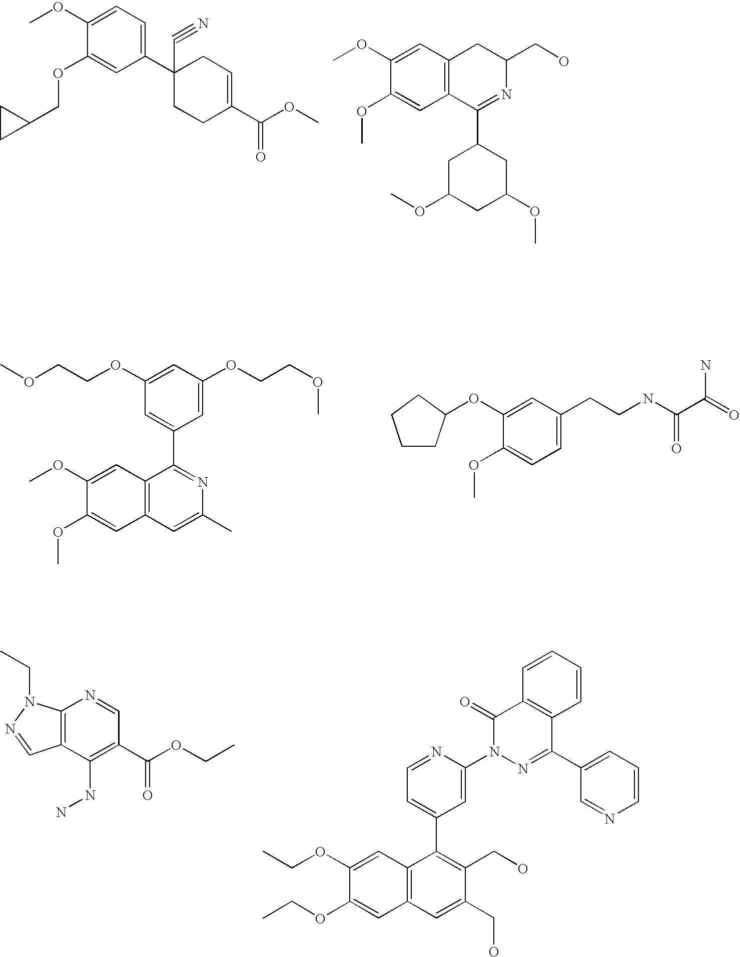

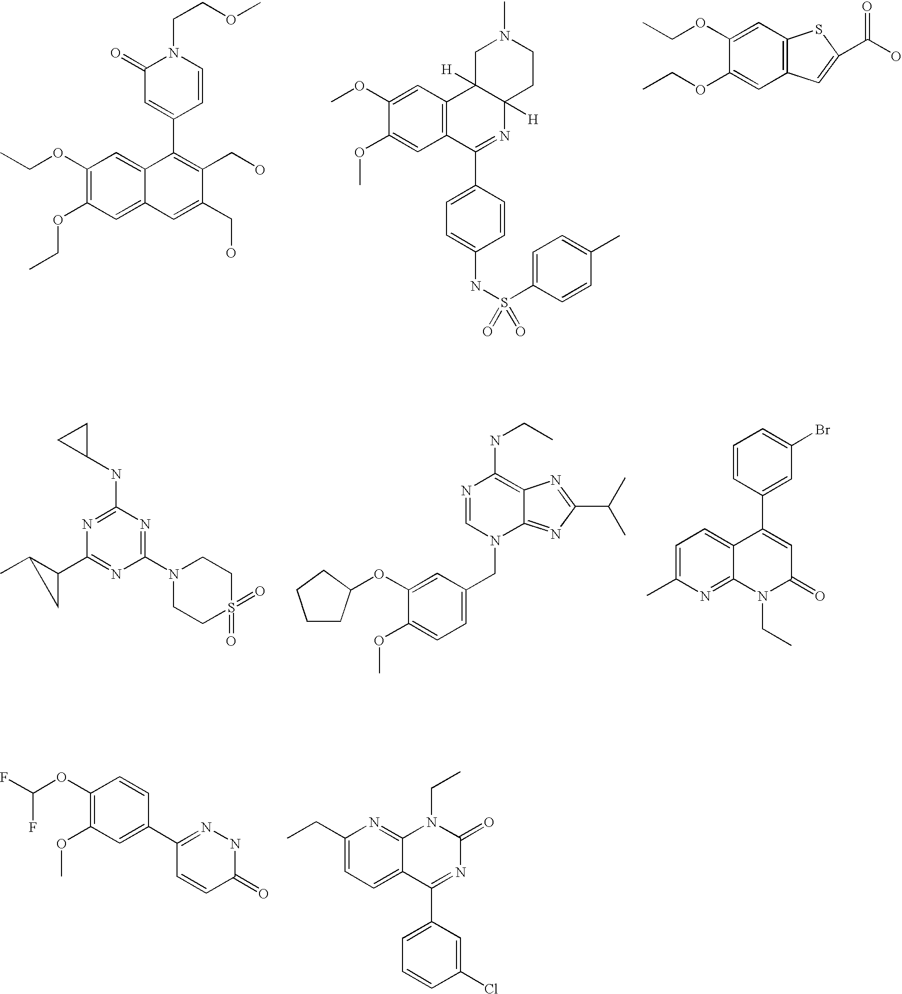

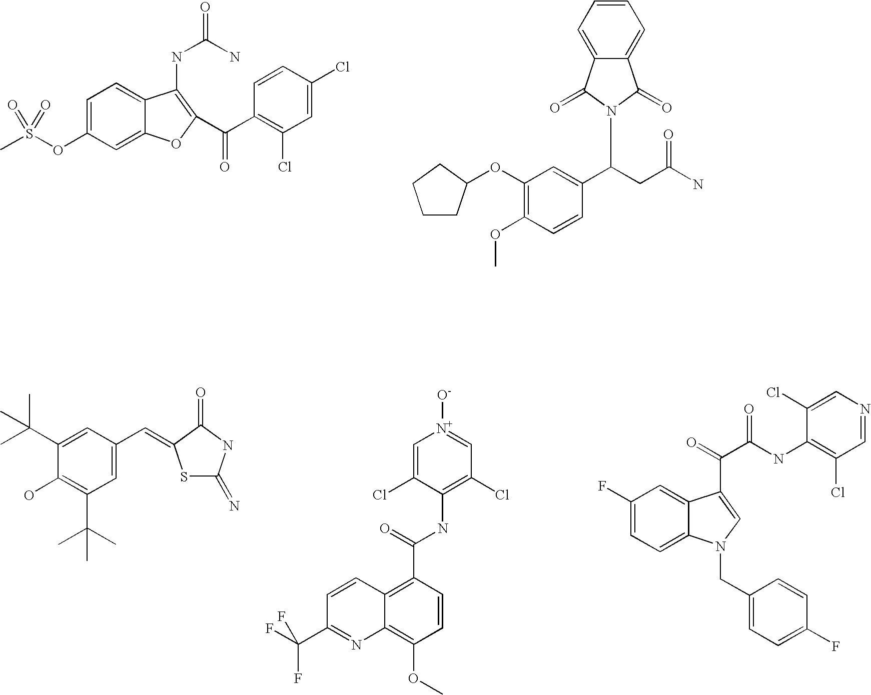

In the above formulae there is given neither any stereochemical information nor are hydrogen atoms indicated [—O is accordingly —OH, >N is >NH or —N is NH₂]. Methyl groups, e.g. on the oxygen atoms, are indicated by lines].

Those PDE4 or PDE3/4 inhibitors are to be emphasized which are named expressis verbis as an example and/or claimed generically in the patent applications or patents EP 0163965, EP 0389282, EP 0393500, EP 0435811, EP 0482302, EP 0499216, EP 0506194, EP 0510562, EP 0528922, EP 0553174, EP 0731099, WO 9319749, WO 9500516, WO 9501338, WO 9600218, WO 9603399, WO 9611690, WO 9636625, WO 9636626, WO 9723457, WO 9728131, WO 9735854, WO 9740032, WO 9743288, WO 9809946, WO 9807715, WO 9808841, WO 9821207, WO 9821208, WO 9821209, WO 9822453, WO 9831674, WO 9840382, WO 9855481, WO 9905111, WO 9905112, WO 9905113, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777, WO 0151470 WO 02006270, WO 02066476, WO 02064584 and WO 02085885 and the compounds with the following research codes: CDC-998, D4396, SCH-351591, IC-485, CC-1088 and KW-4490. Substances having good oral availability are preferred here.

Preferred PDE4 or PDE3/4 inhibitors are 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]-benzo-diazepin-3(R)-yl]pyridine-4-carboxamide [Research-Code: CI-1018], 4-(3,4-dimethoxyphenyl)-thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenyl-carbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and the compounds with the research codes CDC-998, SH-636, D-4396, IC-485, CC-1088 and KW-4490.

Further preferred PDE4 inhibitors are selected from the group consisting of
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁶-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁸-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)piperidin-4-yl]4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxy-phenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)-piperidin-4-yl]4a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl-4-a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one,
(4aS,8aR)-4-(3,4-Diethoxyphenyl)-2-(1 {2-[4-(2-dimethylamino-ethyl)-piperazin-1-yl]-ethanoyl)-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1 {4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester and
2-{4-[(4aS, 8aR)-4-(3,4-Dimethoxyphenyl)-1-oxo-4a,5,8,8a-tetrahydro-1H-phthalazin-2-yl]-piperidin-1-yl}-2H-acetamide.

Particularly preferred PDE4 or PDE3/4 inhibitors are 3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a, 1 b-hexahydro-6-(4-diisopropylaminocarbonylphenyl)-benzo-[c][1,6]naphthyridine [INN: PUMAFENTRINE].

As possible histamine receptor antagonists within the meaning of the present invention may be mentioned, by way of example, (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]-pyridin [INN: AZATADINE], 4-[(4-chlorophenyl)methyl]-2-(hexahydro-1-methyl-1H-azepin-4-yl)-1(2H)-phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE), (plus/minus)-[2-[4-(p-chloro-alpha-phenylbenzyl)-1-piperazin-yl]ethoxy]-acetic acid [INN: CETIRIZINE], (+)-2-2-[(p-Chlor-alpha-methyl-alpha phenylbenzyl)oxy]-ethyl}-1-methylpyrrolidin [INN: CLEMASTINE], 8-chloro-6,11-dihydro-1-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenyl-benzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]-pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and 1-(4-fluorobenzyl)-2-(piperidin-4-ylamino)-1H-benzimidazole [INN: TECASTEMIZOLE].

Preferred histamine receptor antagonists are 4-[(4-chlorophenyl)methyl]-2-(hexahydro-1-methyl-1H-azepin-4-yl)-1 (2H)phthalazinone [INN: AZELASTINE], (plus/minus)-[2-[4-(p-chloro-alpha-phenyl-benzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-1H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

Particularly preferred histamine receptor antagonists are 8-chloro-6,11-dihydro-11-(4-piperidylidene)-5H-benzo[5,6]cyclohepta-[1,2-b]pyridine [INN: DESLORATADINE] and ethyl 4-(8-chloro-5,6-dihydro-11H-benzo[5,6]cyclohepta[1,2-b]pyridin-11-ylidene)-1-piperidinecarboxylate [INN: LORATADINE].

In the context of the present invention, unless otherwise stated, a pharmaceutically acceptable derivative of an active ingredient means a pharmaceutically acceptable salt or solvate (e.g. hydrate), a pharmaceutically acceptable solvate of such salt, a pharmaceutically acceptable N-oxide or a pharmaceutically acceptable salt or solvate of the latter.

Suitable pharmacologically tolerable salts here are on the one hand in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing therefrom. Furthermore, the active compounds mentioned can also be present as pure enantiomers or as enantiomer mixtures in any mixing ratio.

On the other hand, salts with bases are also suitable. Examples of salts with bases which may be mentioned are alkali metal (lithium, sodium, potassium) or calcium, aluminium, magnesium, titanium, ammonium, meglumine or guanidinium salts, where here too the bases are employed in salt preparation in an equimolar quantitative ratio or one differing therefrom.

Certain of the active ingredients used in the present invention are capable of existing in stereoisomeric forms. The invention encompasses all stereoisomers of the active ingredients and mixtures thereof including racemates. Tautomers and mixtures thereof of the active ingredients are also part of the present invention.

In accordance with the present invention, there is provided in a first aspect a pharmaceutical composition comprising, in admixture, a first active ingredient which is selected from a PDE4 inhibitor, a PDE3/4 inhibitor and their pharmaceutically acceptable derivatives, and a second active ingredient which is selected from a histamine receptor antagonist and its pharmaceutically acceptable derivatives.

In a second aspect—which is an embodiment of the first aspect—there is provided a pharmaceutical composition comprising, in admixture, a first active ingredient which is selected from 3-Cyclopropyl-methoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxy-benzyl]-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidinone [INN: DARBUFELONE], cis-[4-Cyano-4-(3-cyclopentyloxy-4-methoxyphenyl)cyclohexane-1-carboxylic acid [INN: CILOMILAST], the compounds with the research codes CDC-998, SH-636, D4396, IC-485, CC-1088, KW-4490 and their pharmaceutically acceptable derivatives, and a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1 (2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]-pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydromethyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11-H-benzo-[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]-pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsuffoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], 1-(4-fluorobenzyl)-2-(piperidin-4-ylamino)-1H-benzimidazole [INN: TECASTEMIZOLE] and their pharmaceutically acceptable derivatives.

In a third aspect—which is another embodiment of the first aspect—there is provided a pharmaceutical composition comprising, in admixture, a first active ingredient which is selected from 3-Cyclopropyl-methoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxy-benzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-di-chloropyridinyl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], 1-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], the compounds with the research codes CDC-998, SH-636, D-4396, IC-485, CC-1088 and their pharmaceutically acceptable derivatives, and a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1 (2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-(2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]-propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE), ethyl 4-(8-chloro-5,6-dihydro-11H-benzo-[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]-pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], I-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and their pharmaceutically acceptable derivatives.

In a forth aspect—which is still another embodiment of the first aspect—there is provided a Pharmaceutical composition comprising, in admixture, a first active ingredient which is selected from 3-Cyclo-propylinethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a, 10b-hexahydro-6-(4-diisopropylaminocarbonylphenylybenzo-[c][1,6]naphthyridine [INN: PUMAFENTRINE] and their pharmaceutically acceptable derivatives, and a second active ingredient which is selected from 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11-H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]-pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE] and their pharmaceutically acceptable derivatives.

In the fifth aspect—which is a further embodiment of the first aspect—there is provided a pharmaceutical composition comprising, in admixture, a first active ingredient which is selected from (cis)-4-(2,3-Dihydro-2,2-dimethyl-7-methoxybenzofuran-4-yl-2-(tetrahydrothiopyran-4-yl-4a,5,8,8a-tetrahydro-2H-phthalazin-1-one, (cis)-4-(3,4-Dimethoxyphenyl)-2-(1,1-dioxohexahydro-10-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1l⁶-thiopyran-4-yl)-4a,5,8,8a-tetrahydro-2H-phthalazin-1-one, (cis)-4-(2,3-Dihydro-2,2-dimethyl-7-methoxybenzofuran-4-yl-2-(1,1-dioxohexahydro-1l⁶-thiopyran-4-yl)-4a,5,8,8a-tetrahydro-2H-phthalazin-1-one, (4aR,8aS)-(cis)-4-(3,4-Dimethoxyphenyl)-2-(1,1-dioxohexahydro-11-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]-4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxyphenyl)-4a,5,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one, (4aS,8aR)-4-(3,4-Dimethoxyphenyl)-2-[1-(4-nitro-phenyl)-piperidin-4-yl-4a,5,8,8a-tetrahydro-2H-phthalazin-1-one, (4aS,8aR)-4-(3,4-Dimethoxyphenyl)-2-(1-pyridin-4-ylmethyl-piperidin-4-yl)-4a,5,8,8a-tetrahydro-2H-phthalazin-1-one, (4aS,8aR)-4-(3,4-Dimethoxyphenyl)-2-[1-(morpholine-4-carbonyl)-piperidin-4-yl]a,5,8,8a-tetrahydro-2H-phthalazin-1-one, (4aS,8aR)-2-{1-[2-(4-Amino-3,5-dichloro-phenyl)-2-oxo-ethyl]-piperidin-4-yl}-4-(3,4-dimethoxy-phenyl)-4a,5,8,8a-tetrahydro-2H-phthalazin-1-one, 4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one, (4aS,8aR)-4-(3,4-Dimethoxyphenyl)-2-(1-pyrimidin-2-yl-piperidin-4-yl)-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-4-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)-2-[1-(morpholine-4-carbonyl)-piperidin-4-yl]4a,5,8,8a-tetrahydro-2H-phthalazin-1-one, (4aS,8aR)-4-(3,4-Dimethoxyphenyl)-2-(1-pyridin-3-ylmethyl-piperidin-4-yl)-4a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)-piperidinyl]-4a,5,8,8a-tetrahydro-2H-phthalazin-1-one, (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide, (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one, 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione, 4-(2-{4-[(4aS,8aR)-4-(3,4-Dimethoxy-phenylyl-oxo-4a,5,8,8a-tetrahydro-1H-phthalazin-2-yl]-piperidin-1-yl}-ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmaceutically acceptable derivatives, and a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE]-4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[_{1,5}-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST],8-chloro-11-[1-(5-methylpyridin-3-ylmethyl)piperidin-4-ylidene]-6,11-dihydro-5H-benzo[5,6]cyclohepta[1,2-b]-pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)ethylenediamine [INN: TRIPELENAMINE], and 1-(4-fluorobenzyl)-2-(piperidin-4-ylamino)-1H-benzimidazole [INN: TECASTEMIZOLE] and their pharmaceutically acceptable derivatives.

In a sixth aspect—which is still a further embodiment of the first aspect—there is provided a pharmaceutical composition comprising, in admixture, a first active ingredient which is selected from (cis)-4-(2,3-Dihydro-2,2-dimethyl-7-methoxybenzofuran-4-yl)-2-(tetrahydrothlopyran-4-yl)-4a,5,8,8a-tetrahydro-2H-phthalazin-1-one, (cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1 l⁶-thiopyran-4-yl)-4a,5,8,8a-tetrahydro-2H-phthalazin-1-one, (cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one, (4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxyphenyl)-4a,5,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one, (4aS,8aR)-4-(3,4-Dimethoxyphenyl)-2-[1-(4-nitro-phenylpiperidin-4-yl]-4a,5,8,8a-tetrahydro-2H-phthalazin-1-one, (4aS,8aR)-4-(3,4-Dimethoxyphenyl)-2-(1-pyridin-4-ylmethyl-piperidin-4-yl)-4a,5,8,8a-tetrahydro-2H-phthalazin-1-one, (4aS,8aR)-4-(3,4-Dimethoxyphenyl)-2-[1-(morpholine-4-carbonyl)-piperidin-4-yl]4a,5,8,8a-tetrahydro-2H-phthalazin-1-one, (4aS,8aR)-2-{1-[2-(4-Amino-3,5-dichloro-phenyl)-2-oxo-ethyl]-piperidin-4-yl}-4-(3,4-dimethoxy-phenyl)-4a,5,8,8a-tetrahydro-2H-phthalazin-1-one, 4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one, (4aS,8aR)-4-(3,4-Dimethoxyphenyl)-2-(1-pyrimidin-2-yl-piperidin-4-yl)-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-4-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)-2-[1-(morpholine-4-carbonyl)-piperidin-4-yl]4a,5,8,8a-tetrahydro-2H-phthalazin-1-one, (4aS,8aR)(3,4-Dimethoxyphenyl)-2-(1-pyridin-3-ylmethyl-piperidin-4-yl)-4a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)-piperidin-4-yl-]4a,5,8,8a-tetrahydro-2H-phthalazin-1-one, (4aS,8aR)-4-(3,4-Diethoxyphenyl)-2-(1-{2-[4-(2-dimethylamino-ethyl)-piperazin-1-yl]-ethanoyl}piperidin-4-yl)-4a,5,8,8a-tetrahydro-2H-phthalazin-1-one, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide, (4aS,8aR)-4-(3,4-Dimethoxyphenyl)-2-[1-(4-1,2,3-thiadiazol-4-yl-benzyl)-piperidin-4-yl]4a,5,8,8a-tetrahydro-2H-phthalazin-1-one, 1-(1-{4-[(4aS,8aR)-4-(3,4-Dimethoxyphenyl)-1-oxo-4a,5,8,8a-tetrahydro-1H-phthalazin-2-yl]-piperidin-1-yl)methanoyl)-4-ethyl-piperazine-2,3-dione, 4-(2-{4[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmaceutically acceptable derivatives, and a second active ingredient which is selected from 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-1-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)-piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1-piperidyl]-butanol [INN: TERFENADINE] and their pharmaceutically acceptable derivatives.

In a seventh aspect the invention provides a pharmaceutical product comprising, in combination, a preparation of a first active ingredient which is selected from a PDE4 inhibitor, a PDE3/4 inhibitor and their pharmaceutically acceptable derivatives, and a preparation of a second active Ingredient which is selected from a histamine receptor antagonist and its pharmaceutically acceptable derivatives, for simultaneous, sequential or separate use in therapy.

In a eighth aspect—which is an embodiment of the seventh aspect—the invention provides a Pharmaceutical product comprising, In combination, a preparation of a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonyl-phenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzylyl H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], the compounds with the research codes CDC-998, SH-636, D-4396, IC-485, CC-1088, KW-4490 and their pharmaceutically acceptable derivatives, and a preparation of a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-[(2H)phthalazinone [INN: AZELASTINE], (+)-(S)-4-[4-[1-(4-chlorophenyl-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-(2-[(p-Chlor-alpha-methyl-alpha phenylbenzyl)oxy]ethyl}-1-methylpyrrolidin [INN: CLEMASTINE], 8-chloro-6,11-dihydro-1-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperdino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]-ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperldinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-1-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], 1-(4-fluorobenzyl)-2-(piperidin-4-ylamino)-1H-benzimidazole [INN: TECASTEMIZOLE] and their pharmaceutically acceptable derivatives, for simultaneous, sequential or separate use in therapy.

In a ninth aspect—which is another embodiment of the seventh aspect—the invention provides a pharmaceutical product comprising, in combination, a preparation of a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], the compounds with the research codes CDC-998, SH-636, D-4396, IC-485, CC-1088 and their pharmaceutically acceptable derivatives, and a preparation of a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]-pyridin [INN: AZATADINE], 4-[(4-chlorophenyl)methyl]-2-(hexahydro-1-methyl-1H-azepin-4-yl)-1(2H)-phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]-ethyl}1-methylpyrrolidin [INN: CLEMASTINE], 8-chloro-6,11-dihydro-1-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinol-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(H-tetrazol-5-yl)-4H-pyrido[1,2-a]pyrimidin-4-one [INN: PEMIROLAST], 8-chloro-1-[1-(5-methylpyridin-3-ylmethyl)piperidin-4-ylidene]-6,11-dihydro-5H-benzo[5,6]-cyclohepta[1,2-b]pyridine [INN: RUPATADINE), 1-[2-[(p-chloro-alpha-methyl-alpha-phenylbenzyl]oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and their pharmaceutically acceptable derivatives, for simultaneous, sequential or separate use in therapy.

In a tenth aspect—which is still another embodiment of the seventh aspect—the invention provides a pharmaceutical product comprising, in combination, a preparation of a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE] and their pharmaceutically acceptable derivatives, and a preparation of a second active ingredient which is selected from 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-1-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE] and their pharmaceutically acceptable derivatives, for simultaneous, sequential or separate use in therapy.

In the eleventh aspect—which is a further embodiment of the seventh aspect—the invention provides a pharmaceutical product comprising, in combination, a preparation of a first active ingredient which is selected from (cis)-4-(2,3-Dihydro-2,2-dimethyl-7-methoxybenzofuran-4-yl)-2-(tetrahydrothiopyran-4-yl)⁴a,5,8,8a-tetrahydro-2H-phthalazin-1-one, (cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1l⁶-thiopyran-4-yl)-4a,5,8,8a-tetrahydro-2H-phthalazin-1-one, (cis)-4-(2,3-Dihydro-2,2-dimethyl-7-methoxybenzofuran-4-yl)-2-(1,1-dioxohexahydro-1l⁵-thiopyran-4-yl)-4a,5,8,8a-tetrahydro-2H-phthalazin-1-one, (4aR,8aS)-(cis)-4-(3,4-Dimethoxyphenyl)-2-(1,1-dioxohexahydro-1 ¹⁶-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]-4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxyphenyl)-4a,5,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one, (4aS,8aR)-4-(3,4-Dimethoxyphenyl)-2-[1-(4-nitro-phenyl)-piperidin-4-yl]-4a,5,8,8a-tetrahydro-2H-phthalazin-1-one, (4aS,8aR)-4-(3,4-Dimethoxyphenyl)-2-(1-pyridin-4-ylmethyl-piperidin-4-yl)-4a,5,8,8a-tetrahydro-2H-phthalazin-1-one, (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one, (4aS,8aR)-2-{1-[2-(4-Amino-3,5-dichloro-phenyl)-2-oxo-ethyl]-piperidin-4-yl}4-(3,4-dimethoxy-phenyl)-4a,5,8,8a-tetrahydro-2H-phthalazin-1-one, 4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one, (4aS,8aR)-4-(3,4-Dimethoxyphenyl)-2-(1-pyrimidin-2-yl-piperidin-4-yl)-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-4-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)-2-[1-(morpholine-4-carbonyl)-piperidin-4-yl]-4a,5,8,8a-tetrahydro-2H-phthalazin-1-one, (4aS,8aR)-4-(3,4-Dimethoxyphenyl)-2-(1-pyridin-3-ylmethyl-piperidin-4-yl)-4a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one, (4aS,8aR)-4-(3,4-Diethoxyphenyl)-2-(1-{2-[4-(2-dimethylamino-ethyl)-piperazin-1-yl]-ethanoyl-3-piperidin-4-yl}4a,5,8,8a-tetrahydro-2H-phthalazin-1-one, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide, (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one, 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione, 4-(2-{4-[(4aS, 8aR)-4-(3,4-Dimethoxy-phenyl)-1-oxo-4a,5,8,8a-tetrahydro-1H-phthalazin-2-yl]-piperidin-1-yl)-ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmaceutically acceptable derivatives, and a preparation of a second active ingredient which is selected (E)-6-[(E)-3-(1-pyrrolidinyl)-1-p-tolylpropenyl]-2-pyridineacrylic acid [INN: ACRIVASTINE], 6,11]-Dihydro-11-(1-methyl-4-piperidyliden)-5H-benzo[5,6]cyclohepta-[1,2-b]-pyridin [INN: AZATADINE], 4-[(4-chlorophenyl)methyl]-2-(hexahydro-1-methyl-1H-azepin-4-yl)-1(2H)-phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-(2-[(p-Chlor-alpha-methyl-alpha phenylbenzyl) oxy]ethyl}1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-d]azepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-1-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]-ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4-(alpha-hydroxydiphenylmethyl)-1′-piperidyl]-butanol [INN: TERFENADINE], N-benzyl-N,N-dimethyl-N-(2-pyridyl)-ethylenediamine [INN: TRIPELENAMINE], and 1-(4-fluorobenzyl)-2-(piperidin-4-ylamino)-1H-benzimidazole [INN: TECASTEMIZOLE] and their pharmaceutically acceptable derivatives, for simultaneous, sequential or separate use in therapy.

In the twelfth aspect, which is a still further embodiment of the seventh aspect—the invention provides a pharmaceutical product comprising, in combination, a preparation of a first active ingredient which is selected from (cis)-4-(2,3-Dihydro-2,2-dimethyl-7-methoxybenzofuran-4-yl)-2-(tetrahydrothlopyran-4-yl)-4a,5,8,8a-tetrahydro-2H-phthalazin-1-one, (cis)-4-(3,4-Dimethoxyphenyl)-2-(1,1-dioxohexahydro-1 l⁶-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1l⁶-thiopyran-4-yl)-4a,5,8,8a-tetrahydro-2H-phthalazin-1-one, (cis)-4-(2,3-Dihydro-2,2-dimethyl-7-methoxybenzofuran-4-yl)-2-(1,1-dioxohexahydro-1l⁸-thiopyran-4-yl)-4a,5,8,8a-tetrahydro-2H-phthalazin-1-one, (4aR,8aS)-(cis)-4-(3,4-Dimethoxyphenyl)-2-(1,1-dioxohexahydro-1l⁸-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxyphenyl)-4a,5,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS,8aR)-4-(3,4-Dimethoxyphenyl)-2-[1-(5-dimethylamino-naphthalene-1-sulfonyl)-piperldinyl]-4a,5,8,8a-tetrahydro-2H-phthalazin-1-one, (4aS,8aR)-4-(3,4-Dimethoxyphenyl)-2-[1-(4-nitro-phenyl)-piperidin-4-yl]-4a,5,8,8a-tetrahydro-2H phthalazin-1-one, (4aS,8aR)-4-(3,4-Dimethoxyphenyl)-2-(1-pyridin-4-ylmethyl-piperidin-4-yl)-4a,5,8,8a-tetrahydro-2H phthalazin-1-one, (4aS,8aR)-4-(3,4-Dimethoxyphenyl)-2-(1-(morpholine-4-carbonyl)-piperidin-4-yl]-4a,5,8,8a-tetrahydro-2H-phthalazin-1-one, (4aS,8aR)-2-{1-[2-(4-Amino-3,5-dichloro-phenyl)-2-oxo-ethyl]-piperidin-4-yl}-4-(3,4-dimethoxy-phenyl)-4a,5,8,8a-tetrahydro-2H-phthalazin-1-one, 4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4-(3,4-Dimethoxyphenyl)-2-(1-thieno[2,3-d]pyrimidin-4-yl)-piperidin-4-yl)-4a,5,8,8a-tetrahydro-2H-phthalazin-1-one, (4aS,8aR)-4-(3,4-Dimethoxyphenyl)-2-(1-pyrimidin-2-yl-piperidin-4-yl)-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)-2-[1-(morpholine-4-carbonyl)-piperidin-4-yl]-4a,5,8,8a-tetrahydro-2H-phthalazin-1-one, (4aS,8aR)-4-(3,4-Dimethoxyphenyl)-2-(1-pyridin-3-ylmethyl-piperidin-4-yl}4a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one, (4aS,8aR)-4-(3,4-Diethoxyphenyl)-2-(1-1-{2-[4-(2-dimethylamino-ethyl)-piperazin-1-yl]-ethanoyl}-piperidin-4-yl)-4a,5,8,8a-tetrahydro-2H-phthalazin-1-one, 2-(4-[(4aS,8aR)-4-(3,4-Dimethoxyphenyl)-1-oxo-4a,5,8,8a-tetrahydro-1H-phthalazin-2-yl]-piperidin-1-yl)-2H-isopropyl-acetamide, (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one, 1-(1-{4-[(4aS,8aR)(3,4-Dimethoxyphenyl)-1-oxo-4a,5,8,8a-tetrahydro-1H-phthalazin-2-yl]-piperidin-1-yl]methanoyl)-4-ethyl-piperazine-2,3-dione, 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmaceutically acceptable derivatives, and a preparation of a second active ingredient which is selected from 4-[(4-chlorophenyl)methyl]-2-(hexahydro-1-methyl-H-azepin 4-yl)-1(2H)phthalazinone (INN: AZELASTINE], (plus/minus)-[2-[4-(p-chloro-alpha-phenylbenzyl)-piperazinyl]ethoxy]-acetic acid (INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-H-benzimidazol-2-yl]-4-piperidinyl]-N-methyl-amino]-pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE] and their pharmaceutically acceptable derivatives, for simultaneous, sequential or separate use in therapy.

In a thirteenth aspect, the invention provides a kit comprising a preparation of a first active ingredient which is selected from a PDE4 inhibitor, a PDE3/4 inhibitor and their pharmaceutically acceptable derivatives, a preparation of a second active ingredient which is selected from a histamine receptor antagonist and its pharmaceutically acceptable derivatives, and instructions for the simultaneous, sequential or separate administration of the preparations to a patient in need thereof.

In a fourteenth aspect—which is an embodiment of the thirteenth aspect—the invention provides a kit comprising a preparation of a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl) H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], 1-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], the compounds with the research codes CDC-998, SH-636, D-4396, IC-485, CC-1088, KW-4490 and their pharmaceutically acceptable derivatives, a preparation of a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1 (2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}1-methylpyrrolidin [INN: CLEMASTINE], 8-chloro-6,11-dihydro-1-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]-cyclohepta[1,2-b]pyridin-11-ylidene}-1-piperidinecarboxylate [INN: LORATADINE], 2-[N-[1-(4-fluorobenzyl) H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], 1-(4-fluorobenzyl)-2-(piperidin-4-ylamino)-1H-benzimidazole [INN: TECASTEMIZOLE] and their pharmaceutically acceptable derivatives, and instructions for the simultaneous, sequential or separate administration of the preparations to a patient in need thereof.

In a fifteenth aspect—which is another embodiment of the thirteenth aspect—the invention provides a kit comprising a preparation of a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST], the compounds with the research codes CDC-998, SH-636, D-4396, IC-485, CC-1088 and their pharmaceutically acceptable derivatives, a preparation of a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1-(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus) [2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tertbutyl-4-[4-(diphenylmethoxy)piperidino]butyrophenone [INN: EBASTINE], [2-[4-[bis(p-fluorophenyl)-methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX), 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and their pharmaceutically acceptable derivatives, and instructions for the simultaneous, sequential or separate administration of the preparations to a patient in need thereof.

In a sixteenth aspect—which is still another embodiment of the thirteenth aspect—the invention provides a kit comprising a preparation of a first active ingredient which is selected from 3-Cyclopropyl-methoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE] and their pharmaceutically acceptable derivatives, a preparation of a second active ingredient which is selected from 4-[(4-chlorophenyl)methyl]-2-(hexahydro-1-methyl-1H-azepin-4-yl)-1 (2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperdino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl) -piperidyl]-butanol [INN: TERFENADINE] and their pharmaceutically acceptable derivatives, and instructions for the simultaneous, sequential or separate administration of the preparations to a patient in need thereof.

In the seventeenth aspect—which is a further embodiment of the thirteenth aspect—the invention provides a kit comprising a preparation of a first active ingredient which is selected from
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1 l⁵-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothlopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁶-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a, 5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl-4-(3,4-diethoxyphenyl)-4a,5,8,8a-tetrahydro-2H-phthalazin-1-one,
5-(4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxy-phenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a, 5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one,
(4aS,8aR)-4-(3,4-Diethoxyphenyl)-2-(1-{2-(4-(2-dimethylamino-ethyl)-piperazin-1-yl]-ethanoyl}-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl)methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmaceutically acceptable derivatives, a preparation of a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]-pyridin [INN: AZATADINE], 4-[(4-chlorophenyl)methyl]-2-(hexahydro-1-methyl-1H-azepin-4-yl)-1 (2 Hz phthalazinone [INN: AZELASTINE), (+)-(S)-4-[4-[1-(4-chlorophenyl)-1-(2-pyridyl)methoxy]piperidin-1-yl]-butanoic acid [INN: BEPOTASTINE], (plus/minus)[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]-ethyl}-1-methylpyrrolidin [INN: CLEMASTINE], 8-chloro-6,11-dihydro-11-(4-piperidylidene-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]Pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]-pyridine [INN: RUPATADINE], 1-[2-[(p-chloro-alpha-methyl-alpha-phenylbenzyl)oxy]ethyl]hexahydro 1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], and i-(4-fluorobenzyl)-2-(piperidin-4-ylamino)-1H-benzimidazole [INN: TECASTEMIZOLE] and their pharmaceutically acceptable derivatives, and instructions for the simultaneous, sequential or separate administration of the preparations to a patient in need thereof.

In the eighteenth aspect—which is a still further embodiment of the thirteenth aspect—the invention provides a kit comprising a preparation of a first active ingredient which is selected from
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁵-thiopyran-4-yl}4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1 la-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-443,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}4-(3,4-dimethoxy-phenyl)-4a,5,8,8a-tetrahydro-2H-phthalazin-1-one,
4-(3,4-Dimethoxyphenyl-2-[1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl)-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-(4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmaceutically acceptable derivatives, a preparation of a second active ingredient which is selected from 4-[(4-chlorophenyl)methyl]-2-(hexahydro-1-methyl-1H-azepin-4-yl)-1 (2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo-[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1-piperidyl]-butanol [INN: TERFENADINE] and their pharmaceutically acceptable derivatives, and instructions for the simultaneous, sequential or separate administration of the preparations to a patient in need thereof.

It has been found that the administration of active ingredients according to the invention is advantageous because it results—in comparison to the administration of a single active ingredient from the PDE4 and PDE3/4 inhibitors or the histamine receptor antagonists class—in a reduced early allergic response and/or in a reduced late inflammatory airway response.

The pharmaceutical composition of the present invention may be prepared by mixing the first active ingredient with the second active ingredient. Therefore, in the nineteenth aspect of the present invention, there is provided a process for the preparation of a pharmaceutical composition which comprises mixing a first active ingredient which is selected from a PDE4 inhibitor, a PDE3/4 inhibitor and their pharmacologically acceptable derivatives, with a second active ingredient which is selected from a histamine receptor antagonist and its pharmacologically acceptable derivatives.

In a twentieth aspect—which is an embodiment of the nineteenth aspect—there is provided a process for the preparation of a pharmaceutical composition which comprises mixing a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxymethoxyphenyl)cyclohexane-1-carboxylic acid [INN: CILOMILAST], the compounds with the research codes CDC-998, SH-636, D-4396, IC-485, CC-1088, KW-4490 and their pharmacologically acceptable derivatives, with a second active ingredient which is selected from (E)-6-[(E)-3-(1-pyrrolidinylyl-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1 (2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]-ethoxy]-acetic acid [INN: CETIRIZINE], (+)-2-(2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]-cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]4-piperldinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,1-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-SH-benzo[5,6]cyclohepta[1,2-b]-pyridine [INN: RUPATADINE],1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], 1-(4-fluorobenzyl)-2-(piperidin-4-ylamino)-1H-benzimidazole [INN: TECASTEMIZOLE] and their pharmacologically acceptable derivatives.

In a twentyfirst aspect—which is another embodiment of the nineteenth aspect—there is provided a process for the preparation of a pharmaceutical composition which comprises mixing a first active ingredient which is selected from 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarbox-amido]pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk][1,4]benzodiazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN:CIPAMFYLLINE], Tetrahydro-5-[4-methoxy-3-[(1S,2S,4R)-2-norbornyloxy]phenyl]-2(1H)-pyrimidone [INN: ATIZORAM], β-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)-cyclohexane-1-carboxylic acid [INN: CILOMILAST], the compounds with the research codes CDC-998, SH-636, D-4396, IC-485, CC-1088 and their pharmacologically acceptable derivatives, with a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-[(2H)phthalazinone [INN: AZELASTINE], (+)-(S)-4-[4-[1-(4-chlorophenyl)-1-(2-pyridyl)-methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]—piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(I 0,11-dihydro-5H-dibenzo[a,d]cyclohepten-5-ylidene)-N-methyl-1-propanamine [INN: NORTRIPTYLINE], 9-methyl-3-(1H-tetrazol-5-yl}4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and their pharmacologically acceptable derivatives.

In a twentysecond aspect—which is still another embodiment of the nineteenth aspect—there is provided a process for the preparation of a pharmaceutical composition which comprises mixing a first active ingredient which is selected from 3-Cyclopropylmethoxy-4-difluoromethoxy-N-(3,5-dichloropyrid-4-yl)-benzamide [INN: ROFLUMILAST], (−)cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE] and their pharmacologically acceptable derivatives, with a second active ingredient which is selected from 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,1-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]-cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE] and their pharmacologically acceptable derivatives.

In a twentythird aspect—which is still another embodiment of the nineteenth aspect—there is provided a process for the preparation of a pharmaceutical composition which comprises mixing a first active ingredient which is selected from
(cis)-4-(2,3-Dihydro-2,2-dimethyl-7-methoxybenzofuran-4-yl)-2-(tetrahydrothlopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-11-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-11-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁸-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]4a,5,8,8a-tetrahydro-2H-phthalazin-1-one,
(4aS,8aR)-2-(1-[2-(4-Amino-3,5-dichloro-phenyl)-2-oxo-ethyl]-piperidin yl}-4-(3,4-dimethoxy-phenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-one,
(4aS,8aR)-4-(3,4-Diethoxyphenyl)-2-[1-(2-morpholin-4-yl-ethanoyl)-piperidin-4-yl]4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmacologically acceptable derivatives, with a second active ingredient which is selected from (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-1(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)[2-[4-(p-chloro-alpha-phenylbenzyl)-1-piperazinyl]ethoxy]-acetic acid [INN: CETIRIZINE], (+)-2-(2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], 1-(4-fluorobenzyl)-2-(piperidin-4-ylamino)-1H-benzimidazole [INN: TECASTEMIZOLE] and their pharmacologically acceptable derivatives.

In a twentyfourth aspect—which is still another embodiment of the nineteenth aspect—there is provided a process for the preparation of a pharmaceutical composition which comprises mixing a first active ingredient which is selected from (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one, (cis)-4-(3,4-Dimethoxyphenyl)-2-(1l⁸-dioxohexahydro-1l⁸-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1l⁶-thiopyran-4-yl)-4a,5,8,8a-tetrahydro-2H-phthalazin-1-one, (cis)-4-(2,3-Dihydro-2,2-dimethyl-7-methoxybenzofuran-4-yl)-2-(1,1-dioxohexahydro-1 l⁸-thiopyran-4-yl)-4a,5,8,8a-tetrahydro-2H-phthalazin-1-one, (4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]-4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxyphenyl)-4a,6,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl]-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one, (4aS,8aR)-4-(3,4-Dimethoxyphenyl)-2-[1-(4-nitro-phenyl)-piperidin-4-yl]-4a,5,8,8a-tetrahydro-2H-phthalazin-1-one, (4aS,8aR)-4-(3,4-Dimethoxyphenyl)-2-(1-pyridin-4-ylmethyl-piperidin-4-yl)-4a,5,8,8a-tetrahydro-2H-phthalazin-1-one, (4aS,8aR)-4-(3,4-Dimethoxyphenyl)-2-[1-(morpholine-4-carbonyl)-piperidin-4-yl]4a,5,8,8a-tetrahydro-2H-phthalazin-1-one, (4aS,8aR)-2-(1-[2-(4-Amino-3,5-dichloro-phenyl)-2-oxo-ethyl]-piperidin-4-yl)-4-(3,4-dimethoxy-phenyl)-4a,5,8,8a-tetrahydro-2H-phthalazin-1-one, 4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4-(3,4-Dimethoxyphenyl)-2-(1-thieno[2,3-d]pyrimidin-4-yl-piperidin-4-yl)-4a, 5,8,8a-tetrahydro-2H-phthalazin-1-one, (4aS,8aR)-4-(3,4-Dimethoxyphenyl)-2-(1-pyrimidin-2-yl-piperidin-4-yl-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-4-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)-2-[1-(morpholine-4-carbonyl)-piperidin-4-yl]-4a,5,8,8a-tetrahydro-2H-phthalazin-1-one, (4aS,8aR)-4-(3,4-Dimethoxyphenyl)-2-(1-pyridin-3-ylmethyl-piperidin-4-yl)-4a,5,8,8a-tetrahydro-2H-phthalazin-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)-piperidin-4-yl]4a,5,8,8a-tetrahydro-2H-phthalazin-1-one, (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one, 2-{4-[(4aS,8aR)-4-(3,4-Dimethoxyphenyl)-1-oxo-4a,5,8,8a-tetrahydro-1H-phthalazin-2-yl]-piperidin-1-yl}2H-isopropyl-acetamide, (4aS,8aR)-4-(3,4-Dimethoxyphenyl)-2-[1-(4-1,2,3-thiadiazol-4-yl-benzyl)-piperidin-4-yl]4a,5,8,8a-tetrahydro-2H-phthalazin-1-one, 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione, 4-(2-(4-[(4aS,8aR)-4-(3,4-Dimethoxy-phenyl)-1-oxo-4a,5,8,8a-tetrahydro-1H-phthalazin-2-yl]-piperidin-1-yl)ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide and their pharmacologically acceptable derivatives, with a second active ingredient which is selected from 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)-piperdino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one. [INN: MIZOLASTINE] and 1-(p-tert-butylphenyl)-4-[4′-(alpha-hydroxydiphenylmethyl)-1′-piperidyl]-butanol [INN: TERFENADINE] and their pharmacologically acceptable derivatives.

The first and second active ingredient may alternatively (other than in admixture as described above) be administered simultaneously, sequentially or separately to treat respiratory diseases. By sequential is meant that the first and second active ingredient are administered one immediately after the other. Separately means a time difference of administration of up to 24 h, preferably up to 12 h.

The present invention further provides the use of a pharmaceutical composition, or pharmaceutical product according to the invention in the manufacture of a medicament for the prophylaxis and/or treatment of a respiratory disease.

A further aspect of the present invention is a method for the treatment of a respiratory disease comprising administering to a patient in need thereof (a) an effective amount of a PDE4 inhibitor, a PDE3/4 inhibitor or a pharmaceutically acceptable derivative thereof and (b) an effective amount of a histamine receptor antagonist or a pharmaceutically acceptable derivative thereof.

Respiratory diseases which may be mentioned are in particular allergen- and inflammation-induced bronchial disorders (bronchitis, obstructive bronchitis, spastic bronchitis, allergic bronchitis, allergic asthma, bronchial asthma, COPD, allergic, seasonal and perennial rhinitis), which can be treated by the combination according to the invention also in the sense of a long-term therapy (if desired with appropriate adjustment of the dosage of the individual components to the needs at the time, for example needs subject to seasonally related variations).

The active ingredients may, and indeed will, as part of the pharmaceutical composition, the Pharmaceutical product or preparation, be used in admixture with one or more pharmaceutically acceptable auxiliaries and/or excipients.

Within the meaning of the present invention, “use” is preferably understood as meaning the oral administration of both active ingredients. The active ingredients also can be administered as a nasal spray, an aerosol, or as an inhaled powder. Further methods of administration, which may be mentioned are the parenteral (e.g. intravenous) and the transdermal administration of the active ingredients.

The invention encompasses on the one hand co-administering both drugs in one delivery form such as a fixed oral combination (putting both active ingredients in one tablet), as an inhaler (putting both active ingredients in the same inhaler) or as a free oral combination (putting both active ingredients in two separate tablets). On the other hand it encompasses also the administration of the drugs in two different delivery forms such as putting the PDE4 inhibitor into tablets and package them with an inhaler that contains the histamine receptor antagonist, or vice versa.

The person skilled in the art is familiar on the basis of his/her expert knowledge with which excipients or auxiliaries are suitable for the desired pharmaceutical composition, product or preparation. In addition to solvents, gel-forming agents, tablet excipients and other active compound carriers, it is possible to use, for example, antioxidants, dispersants, emulsifiers, antifoams, flavor corrigents, preservatives, solubilizers, colorants or permeation promoters and complexing agents (e.g. cyclodextrins).

The pharmaceutical compositions or preparations according to the invention for oral administration are preferably in the form of tablets, coated tablets, capsules, emulsions, suspensions or solutions, the active ingredient content advantageously being between 0.1 and 95% and by appropriate choice of the excipients and the auxiliaries, it being possible to achieve a pharmaceutical administration form precisely tailored to the active ingredient(s) and/or to the desired onset of action (e.g. a sustained release form or an enteric form).

The active ingredients according to the invention are administered by inhalation preferably in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®), Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical transdermal formulations comprise a conventional aqueous or non aqueous vehicle, for example a cream, ointment, lotion or paste, or are in the form of a medicated plaster, patch or membrane.

For the above-mentioned therapeutic uses the dosages administered will, of course, vary with the first and second active ingredients employed, the mode of administration, the treatment desired and the disorder indicated.

However, in general, satisfactory results will be obtained when the total daily dosage of first active ingredient(s), the PDE4 respectively the PDE3/4 inhibitors, when taken oral is in the range from 1-2000 μg/kg of body weight. In the case of the particularly preferred PDE4 inhibitor ROFLUMILAST, the daily dosage is in a range from 1-20 μg/kg of body weight. The daily dosage for the particularly preferred PDE3/4 inhibitor PUMAFENTRINE is in a range from 300-1500 μg/kg of body weight.

The total daily dosage of the second active ingredient(s), the histamine receptor antagonists also can vary within a wide range (0.1-1500 μg/kg of body weight). In the case of the particularly preferred histamine receptor antagonist LORATADINE, the daily dosage when taken oral is in a range from 0.1-0.5 μg/kg of body weight.

1. A pharmaceutical composition comprising, in admixture, a first active ingredient which is selected from a PDE4 inhibitor, a PDE3/4 inhibitor and their pharmaceutically acceptable derivatives, and a second active ingredient which is selected from a histamine receptor antagonist and its pharmaceutically acceptable derivatives.

2. A pharmaceutical composition according to claim 1, wherein the first and/or second active ingredient is in the form of a pharmaceutically acceptable salt, hydrate, solvate, hydrate of a salt, solvate of a salt, N-oxide, salt of an N-oxide, hydrate of an N-oxide or solvate of an N-oxide.

3. A pharmaceutical composition according to claim 1, which is a fixed oral combination.

4. A pharmaceutical composition according to claim 1, which is a dry powder for use in a dry powder inhaler.

5. A pharmaceutical composition according to claim 1, which is an aqueous preparation for nasal administration.

6. A pharmaceutical composition according to claim 1, in which the PDE4 inhibitor, the PDE3/4 inhibitor or their pharmaceutically acceptable derivatives and the histamine receptor antagonist or its pharmaceutically acceptable derivative is combined with a propellant to form a composition which is delivered using a metered dose inhaler.

7. A pharmaceutical composition according to claim 1, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c] [1,6]naphthyridine [INN: PUMAFENTRINE], 3,5-dichloro-4-[8-methoxy-2-(trifluoromethyl)quinolin-5-ylcarboxamido]-pyridine-1-oxide [Research Code: SCH-351591],3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk]-[1,4]benzo-diazepin-3(R)-yl]pyridine-4-carboxamide [Research Code: CI-1018], 4-(3,4-dimethoxyphenyl)thiazole-2-carboxamide oxime [Research Code: ORG20241], 3,7-dihydro-3-(4-chlorophenyl)-1-propyl-1H-purine-2,6-dione [INN: AROFYLLINE], 3-[3(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]-phenyl]-2(1H)-pyrimidone [INN: ATIZORAM], S-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and the compounds with the research codes CDC-998, SH-636, D-4396, IC-485, CC-1088 and KW-4490, and wherein the histamine receptor antagonist is selected from the group consisting of (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]-cyclohepta-[1,2-b]pyridin [INN: AZATADINE], 4-[(4-chlorophenyl)methyl]-2-(hexahydro-1-methyl-1H-azepin-4-yl)-1(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 4′-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)-piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-(4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[41-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and 1-(4-fluorobenzyl)-2-(piperidin-4-ylamino)-1H-benzimidazole [INN: TECASTEMIZOLE].

8. A pharmaceutical composition according to claim 1, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarboxamido]-pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk]-[1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN: AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]phenyl]-2(1H)-pyrimidone [INN: ATIZORAM], 9-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and the compounds with the research codes CDC-998, SH-636, D-4396, IC-485 and CC-1088, and wherein the histamine receptor antagonist is selected from the group consisting of (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]-cyclohepta-[1,2-b]pyridin [INN: AZATADINE], 4-[(4-chlorophenyl)methyl]-2-(hexahydro-1-methyl-1H-azepin-4-yl)-1(2H)phthalazinone [INN: AZELASTINE], (+)-(S)-4-[4-[1-(4-chlorophenyl)-1-(2-pyridyl)-methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 41-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)-piperidino]-butyl]-alpha-methylhydratropic acid [INN: FEXOFENADINE], 3-[4-(8-fluoro-5,11-dihydrobenz-[b]oxepino[4,3-b]pyridin-11-ylidene)-piperidin-1-yl]-propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo-[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo-[5,6]cyclohepta[1,2-b]pyridine [INN: RUPATADINE], 1-[2-[(p-chloro-alpha-methyl-alpha-phenyl-benzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE] and N-benzyl-N,N′-dimethyl-N-(2-pyridyl)-ethylenediamine [INN: TRIPELENAMINE].

9. A pharmaceutical composition according to claim 1, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], and wherein the histamine receptor antagonist is selected from the group consisting of 4-[(4-chlorophenyl)methyl]-2-(hexahydro-1-methyl-1H-azepin-4-yl)-[(2H)phthalazinone [INN: AZELASTINE], (plus/minus)-[2-[4-(p-chloro-alpha-phenylbenzyl)-1-piperazinyl]ethoxy]acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]-pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

10. A pharmaceutical composition according to claim 9, wherein the PDE4 inhibitor or PDE3/4 inhibitor is selected from the group consisting of (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], and the pharmaceutically acceptable salts, hydrates, solvates, hydrates of the salts, solvates of the salts, N-oxides, salts of the N-oxides, hydrates of the N-oxides or solvates of the N-oxides thereof.

11. A pharmaceutical composition according to claim 9, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof.

12. A pharmaceutical composition according to claim 1, wherein the PDE4 inhibitor or PDE3/4 inhibitor is selected from the group consisting of
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁶-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxy-phenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one,
4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide,
and wherein the histamine receptor antagonist is selected from the group consisting of (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-[(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 41-tert-butyl-4-[4-(diphenylmethoxy)piperidino]butyrophenone [INN: EBASTINE], [2-[4-[bis(p-fluorophenyl)methyl]-1-piperazin-yl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)-piperidino]-butyl]-alpha-methylhydratropic acid [INN: FEXOFENADINE], 3-[4-(8-fluoro-5,11-dihydrobenz[b]oxepino-[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one (INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], and 1-(4-fluorobenzyl)-2-(piperidin-4-ylamino)-1H-benzimidazole [INN: TECASTEMIZOLE].

13. A pharmaceutical composition according to claim 1, wherein the PDE4 inhibitor or PDE3/4 inhibitor is selected from the group consisting of
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁶-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a, 5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS, 8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)piperidin-4-yl]-4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl)piperidin-4-yl)-4a,5,8,8a-tetrahydro-2H-phthalazin-1-one,
2-{4-[(4aS,8aR)-4(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide,
and wherein the histamine receptor antagonist is selected from the group consisting of 4-[(4-chlorophenyl)methyl]-2-(hexahydro-1-methyl-1H-azepin-4-yl)-[(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-(N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

14. A pharmaceutical composition according to claim 9, wherein the histamine receptor antagonist is selected from the group consisting of 8-chloro-6,11-dihydro-11-(4-piperidylidene)-5H-benzo[5,6]cyclohepta-[1,2-b]pyridine [INN: DESLORATADINE], ethyl 4-(8-chloro-5,6-dihydro-11H-benzo[5,6]cyclohepta[1,2-b]pyridin-11-ylidene)-1-piperidinecarboxylate [INN: LORATADINE], and pharmaceutically acceptable salts, hydrates, solvates, hydrates of the salts and solvates of the salts thereof.

15. (canceled)

16. A process for the preparation of a pharmaceutical composition as defined in claim 1 which comprises mixing the first active ingredient with the second active ingredient.

17. A pharmaceutical product comprising, in combination, a preparation of a first active ingredient which is selected from a PDE4 inhibitor, a PDE3/4 inhibitor and their pharmaceutically acceptable derivatives, and a preparation of a second active ingredient which is selected from a histamine receptor antagonist and its pharmaceutically acceptable derivatives, for simultaneous, sequential or separate use in therapy.

18. A pharmaceutical product according to claim 17, wherein the first and/or second active ingredient is in the form of a pharmaceutically acceptable salt, hydrate, solvate, hydrate of a salt, solvate of a salt, N-oxide, salt of an N-oxide, hydrate of an N-oxide or solvate of an N-oxide.

19. A pharmaceutical product according to claim 17, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4[8-methoxy-2-(trifluoromethyl)quinolin-5-ylcarboxamido]-pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk]-[1,4]benzo-diazepin-3(R)-yl]pyridine-4-carboxamide [Research Code: CI-1018], 4-(3,4-dimethoxyphenyl)thiazole-2-carboxamide oxime [Research Code: ORG20241], 3,7-dihydro-3-(4-chlorophenyl)-1-propyl-1H-purine-2,6-dione [INN: AROFYLLINE], 3-[3(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]-phenyl]-2(1H)-pyrimidone [INN: ATIZORAM], 9-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and the compounds with the research codes CDC-998, SH-636, D-4396, IC-485, CC-1088 and KW-4490, and wherein the histamine receptor antagonist is selected from the group consisting of (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]-cyclohepta-[1,2-b]pyridin [INN: AZATADINE], 4-[(4-chlorophenyl)methyl]-2-(hexahydro-1-methyl-1H-azepin-4-yl)-1(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 41-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)-piperidino]-butyl]-alpha-methylhydratropic acid [INN: FEXOFENADINE], 3-[4-(8-fluoro-5,11-dihydrobenz[b]oxepino[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-(4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE] and 1-(4-fluorobenzyl)-2-(piperidin-4-ylamino)-1H-benzimidazole [INN: TECASTEMIZOLE].

20. A pharmaceutical product according to claim 17, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], 3,5-dichloro-4-[8-methoxy-2-(trifluoromethyl)quinolin-5-ylcarboxamido]-pyridine-1-oxide [Research Code: SCH-351591], 3-[3-(cyclopentyloxy)-4-methoxybenzyl]-6-(ethylamino)-8-isopropyl-3H-purine [Research-Code: V-11294A], N-[9-methyl-4-oxo-1-phenyl-3,4,6,7-tetrahydropyrrolo[3,2,1-jk]-[1,4]benzo-diazepin-3(R)-yl]pyridine-4-carboxamide [Research-Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN: AROFYLLINE], 3-[3-(Cyclopentyloxy)-4-methoxybenzylamino]-1H-pyrazole-4-methanol, N-(3,5-dichloro-4-pyridinyl)-2-[1-(4-fluorobenzyl)-5-hydroxy-1H-indol-3-yl]-2-oxoacetamide [Research-Code: AWD-12-281], N-(3,5-dichloropyridin-4-yl)-2-[5-fluoro-1-(4-fluorobenzyl)-1H-indol-3-yl]-2-oxoacetamide [Research-Code: AWD-12-343], 8-Amino-1,3-bis(cyclopropylmethyl)xanthine [INN: CIPAMFYLLINE], Tetrahydro-5-[4-methoxy-3-[(1S,2S,4R)-2-norbornyloxy]phenyl]-2(1H)-pyrimidone [INN: ATIZORAM], 9-[3-(Cyclopentyloxy)-4-methoxyphenyl]-1,3-dihydro-1,3-dioxo-2H-isoindole-2-propanamide [Research-Code: CDC-801], Methanesulfonic acid 2-(2,4-dichlorophenylcarbonyl)-3-ureidobenzo-furan-6-yl ester [INN: LIRIMILAST], (Z)-5-(3,5-di-tert-butyl-4-hydroxybenzylidene)-2-imidazothiazolidin-4-one [INN: DARBUFELONE], cis-[4-Cyano-4-(3-cyclopentyloxy-4-methoxyphenyl)cyclohexane-1-carboxylic acid [INN: CILOMILAST] and the compounds with the research codes CDC-998, SH-636, D-4396, IC-485 and CC-1088, and wherein the histamine receptor antagonist is selected from the group consisting of (E)-6-[(E)-3-(1-pyrrolidinyl)-1-p-tolylpropenyl]-2-pyridineacrylic acid [INN: ACRIVASTINE], 6,11-Dihydro-11-(1-methyl-4-piperidyliden)-5H-benzo[5,6]-cyclohepta-[1,2-b]pyridin [INN: AZATADINE], 4-[(4-chlorophenyl)methyl]-2-(hexahydro-1-methyl-1H-azepin-4-yl)-1(2H)phthalazinone [INN: AZELASTINE], (+)-(S)-4-[4-[1-(4-chlorophenyl)-1-(2-pyridyl)-methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 41-tert-butyl-4-[4-(diphenylmethoxy)piperidino]butyrophenone [INN: EBASTINE], [2-[4-[bis(p-fluorophenyl)methyl]-1-piperazinyl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)-piperidino]-butyl]-alpha-methylhydratropic acid [INN: FEXOFENADINE], 3-[4-(8-fluoro-5,11-dihydrobenz-[b]oxepino[4,3-b]pyridin-11-ylidene)-piperidin-1-yl]-propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo-[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one [INN: PEMIROLAST], 8-chloro-11-[1-(5-methylpyridin-3-ylmethyl)piperidin-4-ylidene]-6,11-dihydro-5H-benzo-[5,6]cyclohepta[1,2-b]pyridine [INN: RUPATADINE], 1-[2-[(p-chloro-alpha-methyl-alpha-phenyl-benzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE] and N-benzyl-N,N′-dimethyl-N-(2-pyridyl)-ethylenediamine [INN: TRIPELENAMINE].

21. A pharmaceutical product according to claim 17, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], and wherein the histamine receptor antagonist is selected from the group consisting of 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]-pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

22. A pharmaceutical product according to claim 21, wherein the PDE4 inhibitor or PDE3/4 inhibitor is selected from the group consisting of (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], and the pharmaceutically acceptable salts, hydrates, solvates, hydrates of the salts, solvates of the salts, N-oxides, salts of the N-oxides, hydrates of the N-oxides or solvates of the N-oxides thereof.

23. A pharmaceutical product according to claim 21, wherein the PDE4 inhibitor or PDE3/4 Inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof.

24. A pharmaceutical product according to claim 17, wherein the PDE4 inhibitor or PDE3/4 inhibitor is selected from the group consisting of (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one, (cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1l⁶-thiopyran-4-yl)-4a,5,8,8a-tetrahydro-2H-phthalazin-1-one, (cis)-4-(2,3-Dihydro-2,2-dimethyl-7-methoxybenzofuran-4-yl)-2-(1,1-dioxohexahydro-1l⁶-thiopyran-4-yl)-4a, 5,8,8a-tetrahydro-2H-phthalazin-1-one, (4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a, 5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]-4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxy-phenyl)-4a,5,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one, (4aS,8aR)-4-(3,4-Dimethoxyphenyl)-2-[1-(4-nitro-phenyl)-piperidin-4-yl]-4a,5,8,8a-tetrahydro-2H-phthalazin-1-one, (4aS,8aR)-4-(3,4-Dimethoxyphenyl)-2-(1-pyridin-4-ylmethyl-piperidin-4-yl)-4a,5,8,8a-tetrahydro-2H-phthalazin-1-one, (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one, (4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one, 4-(3,4-Dimethoxyphenyl)-2-(1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one, (4aS,8aR)-4-(3,4-Dimethoxyphenyl)-2-(1-pyrimidin-2-yl-piperidin-4-yl)-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-4-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)-2-[1-(morpholine-4-carbonyl)-piperidin-4-yl]-4a,5,8,8a-tetrahydro-2H-phthalazin-1-one, (4aS,8aR)-4-(3,4-Dimethoxyphenyl)-2-(1-pyridin-3-ylmethyl-piperidin-4-yl)-4a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one, (4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide, (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one, 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione, 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide, and wherein the histamine receptor antagonist is selected from the group consisting of (E)-6-[(E)-3-(1-pyrrolidinyl)-1-p-tolylpropenyl]-2-pyridineacrylic acid [INN:
ACRIVASTINE], 6,11-Dihydro-11-(1-methyl-4-piperidyliden)-5H-benzo[5,6]cyclohepta-[1,2-b]pyridin [INN: AZATADINE], 4-[(4-chlorophenyl)methyl]-2-(hexahydro-1-methyl-1H-azepin-4-yl)-[(2H)phthalazinone [INN: AZELASTINE], (+)-(S)-4-[4-[1-(4-chlorophenyl)-1-(2-pyridyl)methoxy]piperidin-1-yl]-butanoic acid [INN: BEPOTASTINE], (plus/minus)-[2-[4-(p-chloro-alpha-phenylbenzyl)-1-piperazinyl]ethoxy]-acetic acid [INN: CETIRIZINE], (+)-2-{2-[(p-Chlor-alpha-methyl-alpha phenylbenzyl)oxy]ethyl}-1-methylpyrrolidin [INN: CLEMASTINE], 8-chloro-6,11-dihydro-11-(4-piperidylidene)-5H-benzo[5,6]cyclohepta-[1,2-b]pyridine [INN: DESLORATADINE], [3-(4-Chlorophenyl)-3-pyridin-2-yl-propyl]-dimethylamine [INN: DEXCHLORPHENIRAMINE], 41-tert-butyl-4-[4-(diphenylmethoxy)piperidino]butyrophenone [INN: EBASTINE], [2-[4-[bis(p-fluorophenyl)methyl]-1-piperazin-yl]ethoxy]acetic acid [INN: EFLETIRIZINE], 1-(2-ethoxyethyl)-2-(hexahydro-4-methyl-1H-1,4-diazepin-1-yl)-benzimidazole [INN: EMEDASTINE], 3-amino-9,13b-dihydro-1H-dibenz[c,f]imidazo[1,5-a]azepine [INN: EPINASTINE], (plus/minus)-p-[1-hydroxy-4-[4-(hydroxydiphenylmethyl)-piperidino]-butyl]-alpha-methylhydratropic acid [INN: FEXOFENADINE], 3-[4-(8-fluoro-5,11-dihydrobenz[b]oxepino-[4,3-b]pyridin-11-ylidene)-piperidin-1-yl]propionic acid [Research Code: HSR-609], (−)-(3S,4R)-1-[cis-4-cyano-4-(p-fluorophenyl)cyclohexyl]-3-methyl-4-phenylisonipecotic acid [INN: LEVOCABASTINE], [2-[4-[(R)-p-chloro-alpha-phenylbenzyl)-1-piperazinyl]ethoxy]-acetic acid [INN: LEVOCETIRIZINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE], 1-(4-fluorobenzyl)-2-(piperidin-4-ylamino)-1H-benzimidazole [INN: NORASTEMIZOLE], 3-(10,11-dihydro-5H-dibenzo[a,d]cyclohepten-5-ylidene)-N-methyl-1-propanamine [INN: NORTRIPTYLINE], 9-methyl-3-(1H-tetrazol-5-yl)-4H-pyrido[1,2-a]pyrimidin-4-one (INN: PEMIROLAST], 8-chloro-11-[1-(5-methylpyridin-3-ylmethyl)piperidin-4-ylidene]-6,11-dihydro-5H-benzo[5,6]cyclohepta[1,2-b]pyridine [INN: RUPATADINE], 1-[2-[(p-chloro-alpha-methyl-alpha-phenylbenzyl)oxy]ethyl]hexahydro-1H-azepine [INN: SETASTINE], S-(7-carboxy-4-hexyl-9-oxoxanthen-2-yl)-S-methylsulfoximine [INN: SUDEXANOX], 1-(p-tert-butylphenyl)-4-[4′-(alpha-hydroxydiphenylmethyl)-1′-piperidyl]-butanol [INN: TERFENADINE], N-benzyl-N,N′-dimethyl-N-(2-pyridyl)-ethylenediamine [INN: TRIPELENAMINE], and 1-(4-fluorobenzyl)-2-(piperidin-4-ylamino)-1H-benzimidazole [INN: TECASTEMIZOLE].

25. A pharmaceutical product according to claim 17, wherein the PDE4 inhibitor or PDE3/4 inhibitor is selected from the group consisting of (cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one, (cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one, (cis)-4-(3-Chloro-4-methoxyphenyl)-2-(tetrahydrothiopyran-4-yl)-4a,5,8,8a-tetrahydro-2H-phthalazin-1-one, (cis)-4-(3-Chloro-4-methoxyphenyl)-2-(1-oxo-hexahydro-1l⁴-thiopyran-4-yl)-4a,5,8,8a-tetrahydro-2H-phthalazin-1-one, (cis)-4-(3,4-Diethoxyphenyl)-2-(1,1-dioxohexahydro-1l⁶_thiopyran-4-yl)-4a,5,8,8a-tetrahydro-2H-phthalazin-1-one, (cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one, (4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one, (4aS,8aR)-4-(3,4-Diethoxyphenyl)-2-[1-(toluene-4-sulfonyl)-piperidin-4-yl]-4a,5,8,8a-tetrahydro-2H-phthalazin-1-one, (4aS,8aR)-4-(3,4-Diethoxyphenyl)-2-(1-methanesulfonyl-piperidin-4-yl)-4a,5,8,8a-tetrahydro-2H-phthalazin-1-one, (4aS,8aR)-2-(1-Acetyl-piperidin-4-yl)-4-(3,4-diethoxyphenyl)-4a,5,8,8a-tetrahydro-2H-phthalazin-1-one, 5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid, (4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one, 4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide, 4-[(4aS,8aR)-4-(3,4-Diethoxyphenyl)-1-oxo-4a,5,8,8a-tetrahydro-1H-phthalazin-2-yl]-piperidine-1-carboxylic acid phenylamide, (cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide, (4aS, 8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one, (4aS,8aR)-4-(3,4-Dimethoxyphenyl)-2-[1-(4-nitro-phenyl)-piperidin-4-yl]-4a,5,8,8a-tetrahydro-2H-phthalazin-1-one, (4aS,8aR)-4-(3,4-Dimethoxyphenyl)-2-(1-pyridin-4-ylmethyl-piperidin-4-yl)-4a,5,8,8a-tetrahydro-2H-phthalazin-1-one, (4aS,8aR)-4-(3,4-Dimethoxyphenyl)-2-[1-(morpholine-4-carbonyl)-piperidin-4-yl]-4a,5,8,8a-tetrahydro-2H-phthalazin-1-one, (4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one, 4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one, (4aS,8aR)-4(3,4-Dimethoxyphenyl)-2-(1-thieno[2,3-d]pyrimidin-4-yl-piperidin-4-yl)-4a,5,8,8a-tetrahydro-2H-phthalazin-1-one, (4aS,8aR)-4-(3,4-Dimethoxyphenyl)-2-(1-pyrimidin-2-yl-piperidin-4-yl)-4a,5,8,8a-tetrahydro-2H-phthalazin-1-one, (4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one, 4-(3,4-Dimethoxyphenyl)-2-(1-isopropyl-piperidin-4-yl)-4a,5,8,8a-tetrahydro-2H-phthalazin-1-one, (4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one, (4aS,8aR)-4-(3,4-Dimethoxyphenyl)-2-(1-phenethyl-piperidin-4-yl)-4a,5,8,8a-tetrahydro-2H-phthalazin-1-one, (4aS,8aR)-4-(3,4-Diethoxyphenyl)—2-[1-(morpholine-4-carbonyl)-piperidin-4-yl]-4a,5,8,8a-tetrahydro-2H-phthalazin-1-one, (4aS,8aR)-4-(3,4-Dimethoxyphenyl)-2-(1-pyridin-3-ylmethyl-piperidin-4-yl)-4a,5,8,8a-tetrahydro-2H-phthalazin-1-one, (4aS,8aR)-4-(3,4-Dimethoxy-phenyl)-2-(1-pyridin-2-ylmethyl-piperidin-4-yl)-4a,5,8,8a-tetrahydro-2H-phthalazin-1-one, (4aS,8aR)-4-(3,4-Diethoxyphenyl)-2-[1-(2-morpholin-4-yl-ethanoyl)piperidin-4-yl]-4a,5,8,8a-tetrahydro-2H-phthalazin-1-one, (4aS,8aR)-4-(3,4-Diethoxyphenyl)-2-(1-{2-[4-(2-dimethylamino-ethyl)-piperazin-1-yl]-ethanoyl)piperidin-4-yl)-4a,5,8,8a-tetrahydro-2H-phthalazin-1-one, 2-(4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide, (4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one, 1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione, 4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester, 2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide,
and wherein the histamine receptor antagonist is selected from the group consisting of 4-[(4-chlorophenyl)methyl]-2-(hexahydro-1-methyl-1H-azepin-4-yl)-[(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

26. A pharmaceutical product according to claim 21, wherein the histamine receptor antagonist is selected from the group consisting of 8-chloro-6,11-dihydro-11-(4-piperidylidene)-5H-benzo[5,6]cyclohepta-[1,2-b]pyridine [INN: DESLORATADINE], ethyl 4-(8-chloro-5,6-dihydro-11H-benzo[5,6]cyclohepta[1,2-b]pyridin-11-ylidene)-1-piperidinecarboxylate [INN: LORATADINE], and pharmaceutically acceptable salts, hydrates, solvates, hydrates of the salts and solvates of the salts thereof.

27. (canceled)

28. A kit comprising a preparation of a first active ingredient which is selected from a PDE4 inhibitor, a PDE3/4 inhibitor and their pharmaceutically acceptable derivatives, a preparation of a second active ingredient which is selected from a histamine receptor antagonist and its pharmaceutically acceptable derivatives, and instructions for simultaneous, sequential or separate administration to the patient in need thereof.

29. A kit according to claim 28, wherein the first and/or second active ingredient is in the form of a pharmaceutically acceptable salt, hydrate, solvate, hydrate of a salt, solvate of a salt, N-oxide, salt of an N-oxide, hydrate of an N-oxide or solvate of an N-oxide or solvate of an salt or N oxide.

30. A kit according to claim 28, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonyl-phenyl)-benzo-[c][1,6]naphthyridine [INN: PUMAFENTRINE], and wherein the histamine receptor antagonist is selected from the group consisting of 4-[(4-chlorophenyl)methyl]-2-(hexahydro-1-methyl-1H-azepin-4-yl)-[(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]butanol [INN: TERFENADINE].

31. A kit according to claim 30, wherein the PDE4 inhibitor or PDE3/4 inhibitor is selected from the group consisting of (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE], and the pharmaceutically acceptable salts, hydrates, solvates, hydrates of the salts, solvates of the salts, N-oxides, salts of the N-oxides, hydrates of the N-oxides or solvates of the N-oxides thereof.

32. A kit according to claim 30, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof.

33. A kit according to claim 28, wherein the PDE4 inhibitor or PDE3/4 inhibitor is selected from the group consisting of
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one, (cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁶-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS, 8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)piperidin-4-yl]-4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl)piperidin-4-yl)-4a,5,8,8a-tetrahydro-2H-phthalazin-1-one,
2-{4-[(4aS,8aR)-4(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide,
and wherein the histamine receptor antagonist is selected from the group consisting of 4-[(4-chlorophenyl)methyl]-2-(hexahydro-1-methyl-1H-azepin-4-yl)-[(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

34. A kit according to claim 30, wherein the histamine receptor antagonist is selected from the group consisting of 8-chloro-6,11-dihydro-11-(4-piperidylidene)-5H-benzo[5,6]cyclohepta-[1,2-b]pyridine [INN: DESLORATADINE], ethyl 4-(8-chloro-5,6-dihydro-11H-benzo[5,6]cyclohepta[1,2-b]pyridin-11-ylidene)-1-piperidinecarboxylate [INN: LORATADINE], and pharmaceutically acceptable salts, hydrates, solvates, hydrates of the salts and solvates of the salts thereof.

35. A method for the treatment of a respiratory disease comprising administering to a patient in need thereof (a) an effective amount of a PDE4 inhibitor, a PDE3/4 inhibitor or a pharmaceutically acceptable derivative thereof and (b) an effective amount of a histamine receptor antagonist or a pharmaceutically acceptable derivative thereof.

36. A method according to claim 35, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE].

37. A method according to claim 35, wherein the PDE4 inhibitor or PDE3/4 inhibitor is selected from the group consisting of
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyran-4-yl)-4a,5,8,8a-tetrahydro-2H-phthalazin-1-one,
(cis)-4-(3,4-Diethoxyphenyl)-2-(1,1-dioxohexahydro-1l⁶-thiopyran-4-yl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one,
(4aS,8 aR)-4-(3,4-Diethoxyphenyl)-2-[1-(toluene-4-sulfonyl)-piperidin-4-yl]-4a,5,8,8a-tetrahydro-2H-phthalazin-1-one,
(4aS,8 aR)-4-(3,4-Diethoxyphenyl)-2-(1-methanesulfonyl-piperidin-4-yl)-4a,5,8,8a-tetrahydro-2H-phthalazin-1-one,
(4aS, 8aR)-2-(1-Acetyl-piperidin-4-yl)-4-(3,4-diethoxyphenyl)-4a,5,8,8a-tetrahydro-2H-phthalazin-1-one,
5-4-[(4aS,8aR)-4-(3,4-Diethoxy-phenyl)-oxo-4a,5,8,8a-tetrahydro-1H-phthalazin-2-yl]-piperidin-1-yl}-5-oxo-pentanoic acid,
(4aS,8 aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS, 8aR)-4-(3,4-Dimethoxyphenyl)-2-[e-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxyphenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)-piperidin-4-yl]-4a,5,8,8a-tetrahydro-2H-naphthalen-1-one,
(4aS,8aR)-4(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)piperidin-4-yl]-4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl)piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester,
2-{4-[(4aS,8aR)-4-(3,4-Dimethoxyphenyl)-1-oxo-4a,5,8,8a-tetrahydro-1H-phthalazin-2-yl]-piperidin-1-yl}-2H-acetamide.

38. A method according to claim 35, wherein the histamine receptor antagonist is selected from the group consisting of 4-[(4-chlorophenyl)methyl]-2-(hexahydro-1-methyl-1H-azepin-4-yl)-1(2H)phthalazinone [INN: AZELASTINE], (plus/minus)-[2-[4-(p-chloro-alpha-phenylbenzyl)-1-piperazinyl]ethoxy]-acetic acid [INN: CETIRIZINE], 8-chloro-6,11-dihydro-11-(4-piperidylidene)-5H-benzo[5,6]cyclohepta-[1,2-b]pyridine [INN: DESLORATADINE], (plus/minus)-p-[1-hydroxy-4-[4-(hydroxydiphenylmethyl)piperidino]-butyl]-alpha-methylhydratropic acid [INN: FEXOFENADINE], ethyl 4-(8-chloro-5,6-dihydro-11H-benzo[5,6]cyclohepta[1,2-b]pyridin-11-ylidene)-1-piperidinecarboxylate [INN: LORATADINE], 2-[N-[1-(4-fluorobenzyl)-1H-benzimidazol-2-yl]-4-piperidinyl]-N-methyl-amino]pyrimidin-4(3H)-one [INN: MIZOLASTINE] and 1-(p-tert-butylphenyl)-4-[4′-(alpha-hydroxydiphenylmethyl)-1′-piperidyl]-butanol [INN: TERFENADINE].

39. A pharmaceutical composition according to claim 1, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof,
and wherein the histamine receptor antagonist is selected from the group consisting of [2-[4-[(R)-p-chloro-alpha-phenylbenzyl)-1-piperazinyl]ethoxy]-acetic acid [INN: LEVOCETIRIZINE] and its pharmaceutically acceptable derivatives.

40. A pharmaceutical product according to claim 17, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof,
and wherein the histamine receptor antagonist is selected from the group consisting of [2-[4-[(R)-p-chloro-alpha-phenylbenzyl)-1-piperazinyl]ethoxy]-acetic acid [INN: LEVOCETIRIZINE] and its pharmaceutically acceptable derivatives.

41. A pharmaceutical composition according to claim 9, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof, and wherein the histamine receptor antagonist is selected from the group consisting of (plus/minus)-[2-[4-(p-chloro-alpha-phenylbenzyl)-1-piperazinyl]ethoxy]-acetic acid [INN: CETIRIZINE] and its pharmaceutically acceptable derivatives.

42. A pharmaceutical product according to claim 21, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof, and wherein the histamine receptor antagonist is selected from the group consisting of (plus/minus)-[2-[4-(p-chloro-alpha-phenylbenzyl)-1-piperazinyl]ethoxy]-acetic acid [INN: CETIRIZINE] and its pharmaceutically acceptable derivatives.

43. (canceled)

44. A kit according to claim 28, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof,
and wherein the histamine receptor antagonist is selected from the group consisting of [2-[4-[(R)-p-chloro-alpha-phenylbenzyl)-1-piperazinyl]ethoxy]-acetic acid [INN: LEVOCETIRIZINE] and its pharmaceutically acceptable derivatives.

45. A kit according to claim 30, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof, and wherein the histamine receptor antagonist is selected from the group consisting of (plus/minus)-[2-[4-(p-chloro-alpha-phenylbenzyl)-1-piperazinyl]ethoxy]-acetic acid [INN: CETIRIZINE] and its pharmaceutically acceptable derivatives.

46. A method according to claim 35, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof, and wherein the histamine receptor antagonist is selected from the group consisting of [2-[4-[(R)-p-chloro-alpha-phenylbenzyl)-1-piperazinyl]ethoxy]-acetic acid [INN: LEVOCETIRIZINE] and its pharmaceutically acceptable derivatives.

47. A method according to claim 35, wherein the PDE4 inhibitor or PDE3/4 inhibitor is selected from the group consisting of 3-Cyclopropylmethoxy-4-difluoromethoxy-N-(3,5-dichloropyrid-4-yl)-benzamide [INN: ROFLUMILAST], and the pharmaceutically acceptable salts, hydrates, solvates, hydrates of the salts, solvates of the salts, N-oxides, salts of the N-oxides, hydrates of the N-oxides or solvates of the N-oxides thereof, and wherein the histamine receptor antagonist is selected from the group consisting of (plus/minus)-[2-[4-(p-chloro-alpha-phenylbenzyl)-1-piperazinyl]ethoxy]-acetic acid [INN: CETIRIZINE] and its pharmaceutically acceptable derivatives.
